(19) United States
(12) Patent Application Publication
(10) Pub. No.: US 2023/0175062 A1
(43) Pub. Date: Jun. 8, 2023

(54) NANOELECTRIC DEVICES AND USE THEREOF

(71) Applicant: TWIST BIOSCIENCE CORPORATION, South San Francisco, CA (US)

(72) Inventors: Jeremy LACKEY, Foster City, CA (US); Andres FERNANDEZ, San Francisco, CA (US); David DODD, San Francisco, CA (US); Alexander GORYAYNOV, San Francisco, CA (US)

(21) Appl. No.: 18/062,966

(22) Filed: Dec. 7, 2022

Related U.S. Application Data
(60) Provisional application No. 63/287,458, filed on Dec. 8, 2021.
(60) Provisional application No. 63/313,016, filed on Feb. 23, 2022.

(51) Int. Cl. C12Q 1/6874; B01L 3/00

(52) U.S. Cl. CPC C12Q 1/6874; B01L 3/502761; B01L 2300/0645; B01L 2200/0652; B01L 2300/0819

(57) ABSTRACT

Provided herein are compositions, devices, systems and methods for single molecule sensing. Further provided are devices for nucleic acid sequencing. The compositions, devices, systems and methods described herein provide improved retrieval of biomolecule-based information.

### CROSS-REFERENCE

This application claims the benefit of U.S. Provisional Application No. 63/287,458 filed Dec. 8, 2021 and U.S. Provisional Application No. 63/313,016 filed Feb. 23, 2022, which are incorporated by reference in their entirety.

### BACKGROUND

Biomolecule based information storage systems, e.g., DNA-based, have a large storage capacity and stability over time. However, there is a need for scalable, automated, highly accurate and highly efficient systems for generating and reading biomolecules for information storage.

### BRIEF SUMMARY

Provided herein are devices and methods for detection of biomolecules.

Provided herein are methods for single molecule sensing comprising: (a) contacting a molecular sensor with at least one charge modulator, wherein contacting generates a change in current; (b) measuring the change in current; and (c) correlating the change in current with the presence or absence of the at least one charge modulator, thereby sensing the single molecule. Further provided are methods wherein the single molecule comprises a biomolecule. Further provided herein are methods wherein the biomolecule comprises a nucleic acid. Further provided herein are methods wherein the nucleic acid comprises DNA, RNA, or a mixture thereof. Further provided herein are methods wherein the charge sensor comprises a graphene-enabled field effect transistor (GeFET) or CMOS device. Further provided herein are methods wherein the molecular sensor is in electrical communication with a charge sensor. Further provided herein are methods wherein the molecular sensor comprises a polymerase. Further provided herein are methods wherein the molecular sensor comprises an isothermal polymerase. Further provided herein are methods wherein the molecular sensor comprises Phi29 polymerase or variant thereof. Further provided herein are methods wherein the at least one charge modulator comprises a negative or positive charge. Further provided herein are methods wherein the at least one charge modulator comprises a charge modulating nucleotide (CMN). Further provided herein are methods wherein contacting comprising incorporating a CMN into a polynucleotide primer. Further provided herein are methods wherein the CMN comprises at least one modification relative to a canonical nucleotide. Further provided herein are methods wherein the modification comprises a modification to the base of the CMN. Further provided herein are methods wherein the modification comprises a 7-deaza or 8-bromo modified base. Further provided herein are methods wherein the CMN comprises a modification to the 5′ position. Further provided herein are methods wherein the modification comprises a modification to a 5′ polyphosphate or chemical variant thereof. Further provided herein are methods wherein the modification comprises a thiolated or bromated phosphate. Further provided herein are methods wherein the polyphosphate comprises at least 3, 4, 5, 6, 8, or 10 phosphates or variants thereof. Further provided herein are methods wherein the modification comprises a modification to a terminal 5′ polyphosphate or chemical variant thereof. Further provided herein are methods wherein the modification comprises a polymer. Further provided herein are methods wherein the polymer comprises one or more of a nucleic acid chain, a peptide chain, a polysaccharide, a lipid, a synthetic polymer, and a dendrimer. Further provided herein are methods wherein the nucleic acid chain comprises 25-5000 bases. Further provided herein are methods wherein the nucleic acid chain is branched (dendrimeric). Further provided herein are methods wherein the nucleic acid chain comprises a secondary structure. Further provided herein are methods wherein the secondary structure comprises one or more of a hairpin, a loop, a helix, a G-quadraplex, and an I-motif. Further provided herein are methods wherein the nucleic acid chain comprises a single strand, double strand, or triple strand. Further provided herein are methods wherein the nucleic acid chain comprises at least one charge modulating chemical modification. Further provided herein are methods wherein the at least one charge modulating chemical modification increases the charge of the CMN relative to an unmodified nucleotide. Further provided herein are methods wherein the at least one charge modulating chemical modification comprises one or more of an amine, an alkylamine, a guanidinium, a quaternary amine, an imidazolium, a pyridinium, and a pyrrolidinium. Further provided herein are methods wherein the at least one charge modulating chemical modification decreases the charge of the CMN relative to an unmodified nucleotide. Further provided herein are methods wherein the at least one charge modulating chemical modification comprises one or more of a phosphate, a phosphite, a sulfonate, a sulfite, a carboxylate, a xanthate, a thiocarboxylic acid, a boranophosphonate, and a boric acid. Further provided herein are methods wherein the nucleic acid chain comprises at least one sugar-modified nucleotide. Further provided herein are methods wherein the sugar-modified nucleotide comprises a deoxy or dideoxy nucleotide. Further provided herein are methods wherein the nucleic acid chain comprises a DNA-DNA, DNA-RNA, or DNA-PNA hybrid. Further provided herein are methods wherein the nucleic acid chain comprises a phosphate modification. Further provided herein are methods wherein the phosphate modification comprises a hydrophobic group. Further provided herein are methods wherein the hydrophobic group comprises a straight or branched alkyl chain. Further provided herein are methods wherein the phosphate modification comprises a hydrophilic group. Further provided herein are methods wherein the hydrophilic group comprises polyethylene glycol. Further provided herein are methods wherein the polyethylene glycol comprises a molecular weight of 1000-100,000 daltons. Further provided herein are methods wherein the peptide chain is 1-100 amino acids in length. Further provided herein are methods wherein the CMN comprises a charged small molecule. Further provided herein are methods wherein the charged small molecule comprises one or more of a chelator, a dye, and a metal complex. Further provided herein are methods wherein the metal complex comprises a ferrocene, Ru-dipy, and bis-cyclopentadienyl diiron. Further provided herein are methods wherein the change in current is 1 nanoamp to 100 picoamps. Further provided herein are methods wherein the change in current is 100 picoamps to 1 microamp. Further provided herein are methods wherein the change in current is at least 1.01-3 times the background current. Further provided herein are methods wherein steps a-c are repeated at least 50 times. Further provided herein are methods wherein method is configured to detect 1-200 biomolecules per second.

Provided herein chemically-sensitive field effect transistor devices for sensing single molecules comprising: a solid support, wherein the solid support comprises a plurality of loci, wherein each loci comprises: a graphene layer; a gate electrode and a drain electrode, where the gate electrode and the drain electrode are in electrical communication via the graphene layer; and at least one insulating layer, where the insulating layer is located between the gate electrode and the drain electrode; wherein the loci have a pitch of 50-1000 nanometers. Further provided herein are devices wherein the device further comprises at least one ground shield. Further provided herein are devices wherein the at least one ground shield is at ground potential. Further provided herein are devices wherein the device further comprises at least one buried gate. Further provided herein are devices wherein the at least one ground shield comprises an opening which permits electrical communication between the graphene layer and the least one buried gate. Further provided herein are devices wherein the graphene layer is approximately one atom thick. Further provided herein are devices wherein the device comprises 100 to 1 billion loci. Further provided herein are devices wherein each loci is 50-200 nm in size. Further provided herein are devices wherein each loci is a well, channel, or is substantially planer. Further provided herein are devices wherein the device is 4 to 2000 mm². Further provided herein are devices wherein the device is 4 to 16 mm². Further provided herein are devices wherein the device is 200 to 900 mm². Further provided herein are devices wherein the device is 4 to 900 mm².

Provided herein are methods for single molecule polynucleotide sequencing comprising: a) contacting a plurality of polynucleotides with at least one primer and at least one polymerase to form a plurality of ternary complexes, wherein the ternary complex comprising a graphene binder; b) detecting in real time one or more bases of the polynucleotides, wherein detection occurs when the plurality of ternary complexes are bound to the graphene layer described herein; c) removing the ternary complexes from the surface; and d) repeating steps a-c to sequence the polynucleotides. Further provided herein are methods wherein the graphene binder comprises an aromatic group. Further provided herein are methods wherein the graphene binder comprises an aryl or heteroaryl group. Further provided herein are methods wherein the graphene binder comprises a C6-C30 aryl or heteroaryl group. Further provided herein are methods wherein the graphene binder comprises an aromatic hydrocarbon. Further provided herein are methods wherein the graphene binder comprises naphthalene, biphenyl, fluorene, anthracene, phenanthrene, tetracene, chrysene, triphenylene, pyrene, pentacene, perylene, benzo[a]pyrene, corannulene, benzo[ghi]perylene, coronene, ovalene, or benzo[c]fluorene. Further provided herein are methods wherein step c) comprises washing the surface. Further provided herein are methods wherein the ternary complex is attached to the graphene binder via the primer, the polymerase, or the polynucleotide library. Further provided herein are methods wherein the ternary complex is attached via a linker. Further provided herein are methods wherein the ternary complex is attached via a linker using a conjugation. Further provided herein are methods wherein the conjugation comprises nucleophile/carbonyl; an azide/phosphine; 1,4 Michael addition, 1,3-dipolar cycloaddition, inverse electron demand cycloaddition; olefin metathesis; or cross-coupling reaction. Further provided herein are methods wherein removing comprises contacting the surface with a solvent. Further provided herein are methods wherein the solvent comprises an organic solvent. Further provided herein are methods wherein the organic solvent comprises MeCN, methanol, ethanol, 2-propanol, acetone, DMF, formamide, THF, or DMSO. Further provided herein are methods wherein the organic solvent is heated. Further provided herein are methods wherein the polymerase comprises a Phi29 polymerase or variant thereof. Further provided herein are methods wherein the polymerase is configured for incorporation charge modified nucleotides described herein. Further provided herein are methods wherein the polymerase is bound to the surface in step a). Further provided herein are methods wherein the polymerase is not bound to the surface in step a). Further provided herein are methods wherein the plurality of polynucleotides comprises at least 100,000 unique polynucleotides. Further provided herein are methods wherein the plurality of polynucleotides are 50-30,000 bases in length. Further provided herein are methods wherein detecting comprises contacting the ternary complexes with at least one nucleotide. Further provided herein are methods wherein detecting comprises measuring a change in current when a CMN is incorporated. Further provided herein are methods wherein the buried gate has a positive potential during step (a). Further provided herein are methods wherein the buried gate has a positive or negative potential during step (b).

Provided herein are devices for molecular sensing comprising: a first electrode, wherein the first electrode comprises a neck region; a passive layer; a second electrode, and wherein the first electrode and the second electrode are located above the first base layer; wherein a first portion of the neck region overlaps a first portion of the second electrode, such that the first electrode and the second electrode are separated by a nanogap; wherein a second portion of the first electrode and a second portion of the second electrode are separated by the passive layer; and a first base layer and a second base layer, wherein the first base layer is located above the second base layer, and the first electrode and the second electrode are located above the base layers. Further provided herein are devices wherein the nanogap is 1-50 nm. Further provided herein are devices wherein the nanogap is 10-30 nm. Further provided herein are devices wherein the nanogap is no more than 50 nm. Further provided herein are devices wherein the passive layer comprises an oxide. Further provided herein are devices wherein the oxide comprises silicon, nitride, or carbide. Further provided herein are devices wherein the first electrode and the second electrode comprise platinum, titanium nitride, or titanium. Further provided herein are devices wherein the first electrode and the second electrode each comprise a superficial layer of gold. Further provided herein are devices wherein the layer of gold is no more than 75 angstroms thick. Further provided herein are devices wherein the first base layer comprises silicon oxide or silicon nitride. Further provided herein are devices wherein the second base layer comprises silicon. Further provided herein are devices wherein the device further comprises a charge sensor, wherein the charge sensor is in electrical communication with the first electrode and the second electrode. Further provided herein are devices wherein the charge sensor is attached to the first electrode and the second electrode. Further provided herein are devices wherein the charge sensor comprises a polymer. Further provided herein are devices wherein the polymer comprises at least one nucleic acid, amino acid, sugar, or lipid. Further provided herein are devices wherein the charge sensor comprises carbon. Further provided herein are devices wherein the charge sensor is attached to at least one of the first electrode or the second electrode via sulfur-gold interaction. Further provided herein are devices wherein the charge sensor is further attached to a molecular sensor via a tether. Further provided herein are devices wherein the molecular sensor comprises an enzyme. Further provided herein are devices wherein the molecular sensor comprises an antibody. Further provided herein are devices wherein the enzyme comprises a polymerase. Further provided herein are devices wherein the longest linear dimension of the second electrode is perpendicular to the neck region. Further provided herein are devices wherein the longest linear dimension of the second electrode is parallel to the neck region. Further provided herein are devices wherein at least one edge of the first electrode is undercut relative to the passive layer. Further provided herein are devices wherein the surface area of the first electrode is less than the surface area of the second electrode. Further provided herein are devices wherein the longest linear dimension of the first electrode is perpendicular to the longest linear dimension of the neck region. Further provided herein are devices wherein the first electrode and the second electrode each comprise a superficial layer of gold. Further provided herein are devices wherein the first base layer comprises silicon oxide or silicon nitride. Further provided herein are devices wherein the second base layer comprises silicon. Further provided herein are devices wherein the neck region has a width of no more than 200 nm.

Provided herein are devices for molecular sensing comprising: a first electrode, wherein the first electrode is located above the first base layer; a second electrode, wherein the second electrode comprises a neck region; wherein a first portion of the neck region overlaps a first portion of the first electrode, such that the first electrode and the second electrode are separated by a nanogap; wherein a second portion of the first electrode and a second portion of the second electrode are separated by a passive layer; a first base layer and a second base layer, wherein the first base layer is located above the second base layer, and the first electrode and the second electrode are located above the base layers. Further provided herein are devices wherein the passive layer is configured to overlap with a portion of the second electrode. Further provided herein are devices wherein the passive layer is configured to passivate electrode traces. Further provided herein are devices wherein the nanogap is 1-50 nm. Further provided herein are devices wherein the nanogap is 10-30 nm. Further provided herein are devices wherein the nanogap is no more than 50 nm. Further provided herein are devices wherein the passive layer comprises an oxide. Further provided herein are devices wherein the oxide comprises silicon, nitride, or carbide. Further provided herein are devices wherein the first electrode and the second electrode comprise platinum, titanium nitride, or titanium. Further provided herein are devices wherein the first electrode and the second electrode each comprise a superficial layer of gold. Further provided herein are devices wherein the layer of gold is no more than 75 angstroms thick. Further provided herein are devices wherein the first base layer comprises silicon oxide or silicon nitride. Further provided herein are devices wherein the second base layer comprises silicon. Further provided herein are devices wherein the device further comprises a charge sensor, wherein the charge sensor is in electrical communication with the first electrode and the second electrode. Further provided herein are devices wherein the charge sensor is attached to the first electrode and the second electrode. Further provided herein are devices wherein the charge sensor comprises a polymer. Further provided herein are devices wherein the polymer comprises at least one nucleic acid, amino acid, sugar, or lipid. Further provided herein are devices wherein the charge sensor comprises carbon. Further provided herein are devices wherein the charge sensor is attached to at least one of the first electrode or the second electrode via sulfur-gold interaction. Further provided herein are devices wherein the charge sensor is further attached to a molecular sensor via a tether. Further provided herein are devices wherein the molecular sensor comprises an enzyme. Further provided herein are devices wherein the molecular sensor comprises an antibody. Further provided herein are devices wherein the enzyme comprises a polymerase. Further provided herein are devices wherein the longest linear dimension of the second electrode is perpendicular to the neck region. Further provided herein are devices wherein the longest linear dimension of the second electrode is parallel to the neck region. Further provided herein are devices wherein at least one edge of the first electrode is undercut relative to the passive layer. Further provided herein are devices wherein the surface area of the first electrode is less than the surface area of the second electrode. Further provided herein are devices wherein the longest linear dimension of the first electrode is perpendicular to the longest linear dimension of the neck region. Further provided herein are devices wherein the first electrode and the second electrode each comprise a superficial layer of gold. Further provided herein are devices wherein the first base layer comprises silicon oxide or silicon nitride. Further provided herein are devices wherein the second base layer comprises silicon. Further provided herein are devices wherein the neck region has a width of no more than 200 nm.

Provided herein are devices for molecular sensing comprising: a first electrode, wherein the first electrode comprises a neck region; a passive layer, wherein the passive layer comprises a channel or well, where the bottom of the well or channel comprises the first base layer; a second electrode, and wherein the first electrode and the second electrode are located above the first base layer, and the second electrode is at least partially embedded in the passive layer; wherein a first portion of the neck region overlaps a first portion of the second electrode, such that the first electrode and the second electrode are separated by a nanogap; wherein a second portion of the first electrode and a second portion of the second electrode are separated by the passive layer; and a first base layer and a second base layer, wherein the first base layer is located above the second base layer, and the first electrode and the second electrode are located above the base layers. Further provided herein are devices wherein the nanogap is 1-50 nm. Further provided herein are devices wherein the nanogap is 10-30 nm. Further provided herein are devices wherein the nanogap is no more than 50 nm. Further provided herein are devices wherein the passive layer comprises an oxide. Further provided herein are devices wherein the oxide comprises silicon, nitride, or carbide. Further provided herein are devices wherein the first electrode and the second electrode comprise platinum, titanium nitride, or titanium. Further provided herein are devices wherein the first electrode and the second electrode each comprise a superficial layer of gold. Further provided herein are devices wherein the layer of gold is no more than 75 angstroms thick. Further provided herein are devices wherein the first base layer comprises silicon oxide or silicon nitride. Further provided herein are devices wherein the second base layer comprises silicon. Further provided herein are devices wherein the device further comprises a charge sensor, wherein the charge sensor is in electrical communication with the first electrode and the second electrode. Further provided herein are devices wherein the charge sensor is attached to the first electrode and the second electrode. Further provided herein are devices wherein the charge sensor comprises a polymer. Further provided herein are devices wherein the polymer comprises at least one nucleic acid, amino acid, sugar, or lipid. Further provided herein are devices wherein the charge sensor comprises carbon. Further provided herein are devices wherein the charge sensor is attached to at least one of the first electrode or the second electrode via sulfur-gold interaction. Further provided herein are devices wherein the charge sensor is further attached to a molecular sensor via a tether. Further provided herein are devices wherein the molecular sensor comprises an enzyme. Further provided herein are devices wherein the molecular sensor comprises an antibody. Further provided herein are devices wherein the enzyme comprises a polymerase. Further provided herein are devices wherein the longest linear dimension of the second electrode is perpendicular to the neck region. Further provided herein are devices wherein the longest linear dimension of the second electrode is parallel to the neck region. Further provided herein are devices wherein at least one edge of the first electrode is undercut relative to the passive layer. Further provided herein are devices wherein the surface area of the first electrode is less than the surface area of the second electrode. Further provided herein are devices wherein the longest linear dimension of the first electrode is perpendicular to the longest linear dimension of the neck region. Further provided herein are devices wherein the first electrode and the second electrode each comprise a superficial layer of gold. Further provided herein are devices wherein the first base layer comprises silicon oxide or silicon nitride. Further provided herein are devices wherein the second base layer comprises silicon. Further provided herein are devices wherein the neck region has a width of no more than 200 nm.

Provided here are arrays of any one of devices described herein wherein at least some of the first electrodes and second electrodes are independently addressable. Further provided herein are arrays wherein the pitch distance of the nanogaps of at least some of the devices is no more than 200 nanometers. Further provided herein are arrays wherein at least some of the first electrode and second electrode are independently addressable. Further provided herein are arrays wherein at least some of the first electrode and second electrode are independently addressable on an x-y axis. Further provided herein are arrays wherein at least some of the first electrode and second electrode are independently addressable on a z axis. Further provided herein are arrays wherein the array comprises at least 50 devices of any one of the devices described herein. Further provided herein are arrays wherein the array comprises at least 5000 devices of any one of the devices described herein. Further provided herein are arrays wherein the array comprises at least 100,000 devices of any one of the devices described herein. Further provided herein are arrays wherein the pitch distance of the nanogaps of at least some of the devices is no more than 2 micron. Further provided herein are arrays wherein the array further comprises a plurality of vias, wherein the plurality of vias are configured to connect at least two vertical layers of the device. Further provided herein are arrays wherein the array further comprises a plurality of routing connections, wherein the plurality of routing connections are configured for addressable control of each device in the array.

Provided here are methods of fabricating any one of the devices described herein, wherein the method comprises: a) providing one or more base layers; b) depositing material to generate a second electrode; c) patterning the second electrode; d) optionally planarizing; e) depositing material to generate a passive layer; f) depositing material to generate a first electrode; g) patterning the first electrode; and h) isotropically etching the passive layer, such that the edges of the first electrode are undercut. Further provided herein are methods wherein the method further comprises depositing gold on the first top layer of the device. Further provided herein are methods wherein the one or more base layers comprise thermal oxide on silicon. Further provided herein are methods wherein the method comprises etching or lithography. Further provided herein are methods wherein the method comprises RIE (reactive ion etching). Further provided herein are methods wherein patterning comprises lithography and/or RIE. Further provided herein are methods wherein the method does not comprise e-beam or DUV (deep ultraviolet light) lithography. Further provided herein are methods wherein the method comprises deposition of gold on the first electrode and the second electrode. Further provided herein are methods wherein the first electrode and the second electrode are separated by a nanogap. Further provided herein are methods wherein the nanogap is 1-50 nm. Further provided herein are methods wherein the nanogap is 10-30 nm. Further provided herein are methods wherein the nanogap is no more than 50 nm. Further provided herein are methods wherein the passive layer comprises an oxide. Further provided herein are methods wherein the oxide comprises silicon, nitride, or carbide. Further provided herein are methods wherein the first electrode and the second electrode comprise platinum, titanium nitride, or titanium.

Provided herein are methods of using any one of the devices described herein for molecular sensing, comprising: a. providing an analyte; b. reacting, binding, or other allowing the analyte to interact with the sensor; and c. measuring an electrical signal generated from the sensor. Further provided herein are methods wherein the method comprises: a. providing at least one nucleotide triphosphate, at least one template, and at least one primer; b. extending the primer by the at least one nucleotide triphosphate; and c. measuring an electrical signal generated from the polymerase. Further provided herein are methods wherein the method further comprises analyzing the electrical signal to establish the identity of the at least one nucleotide triphosphate. Further provided herein are methods wherein the at least one nucleotide triphosphate comprises a non-canonical base. Further provided herein are methods wherein the at least one nucleotide triphosphate comprises a terminator which is configured to prevent chain extension. Further provided herein are methods wherein the method is repeated to establish the identity of at least 20 bases. Further provided herein are methods wherein the method is repeated to establish the identity of at least 100 bases. Further provided herein are methods wherein the method is repeated to establish the identity of at least 1000 bases.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a non-limiting example of a scheme for polynucleotides synthesis and sequencing according to some embodiments.

FIG. 2 illustrates a non-limiting example of a graphene device described herein according to some embodiments.

FIG. 3 depicts an example of a charge-modulating nucleotide for use with the devices and methods described herein according to some embodiments.

FIG. 4A depicts a top view of a graphene device for polynucleotide sequencing having a buried gate and shield electrode according to some embodiments.

FIG. 4B depicts a side view of a graphene device for polynucleotide sequencing having a buried gate and shield electrodes according to some embodiments.

FIGS. 5A-5C depict a zoom in of a flexible structure, having spots, channels, or wells, respectively, according to some embodiments.

FIG. 6A is a schema of solid support comprising an active area and fluidics interface according to some embodiments; and

FIG. 6B is a front side of an example of a solid support array according to some embodiments. Such arrays in some instances may comprise thousands or millions of polynucleotide synthesis devices as described herein;

FIG. 6C is a back side of an example of a solid support array according to some embodiments;

FIG. 6D is an example of rack-style instrument according to some embodiments. Such instruments may comprise hundreds or thousands of solid support arrays.

FIG. 7 illustrates an example of a computer system according to some embodiments.

FIG. 8 is a block diagram illustrating architecture of a computer system according to some embodiments.

FIG. 9 is a diagram demonstrating a network configured to incorporate a plurality of computer systems, a plurality of cell phones and personal data assistants, and Network Attached Storage (NAS) according to some embodiments.

FIG. 10 is a block diagram of a multiprocessor computer system using a shared virtual address memory space according to some embodiments.

FIG. 11 depicts a nanoelectric device according to some embodiments. An analyte (nucleic acid shown as an example only) is in communication with a molecular sensor connected to the charge sensor which spans a loci.

FIG. 12A depicts a top view of an edge finger device according to some embodiments.

FIG. 12B depicts a side view of an edge finger device according to some embodiments.

FIG. 12C depicts a side view of an edge finger device according to some embodiments. The dotted line indicates a coating, and a nanowire is depicted as bridging two electrodes.

FIG. 13A depicts a top view of a crossed fingers device according to some embodiments.

FIG. 13B depicts a side view of a crossed fingers device according to some embodiments.

FIG. 14A depicts a top view of a crossed fingers with mask device according to some embodiments.

FIG. 14B depicts a side view of a crossed fingers with mask device according to some embodiments.

FIG. 15 depicts an array of multiple crossed fingers device according to some embodiments.

FIG. 16A depicts a top view of a self-aligned finger etch device according to some embodiments.

FIG. 16B depicts a side view of a self-aligned finger etch device according to some embodiments.

### DETAILED DESCRIPTION OF THE INVENTION

There is a need for larger capacity storage systems as the amount of information generated and stored is increasing exponentially. A biomolecule such as a DNA molecule provides a suitable host for information storage in-part due to its stability over time and capacity for four bit (or other) information coding, as opposed to traditional binary information coding. Provided herein are methods, devices, and systems for real-time sensing of single molecules (e.g., biomolecules) Provided herein are methods to improve nucleic acid sequencing through use of nanoelectric sensing devices.

Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects are described herein primarily with regard to the preparation of nucleic acids molecules, the sequence of the polynucleotide being known and chosen before the synthesis or assembly of the nucleic acid molecules.

As used herein, the term “symbol,” generally refers to a representation of a unit of digital information. Digital information may be divided or translated into one or more symbols. In an example, a symbol may be a bit and the bit may have a numerical value. In some examples, a symbol may have a value of ‘0’ or ‘1’. In some examples, digital information may be represented as a sequence of symbols or a string of symbols. In some examples, the sequence of symbols or the string of symbols may comprise binary data.

Polynucleotide sequences described herein may be, unless stated otherwise, comprise DNA or RNA or an analog or derivative thereof. As used herein, the terms nucleic acids, polynucleotides, oligonucleotides, oligos, oligonucleic acids are used synonymously throughout to represent a polymer of nucleoside monomers. In some instances, nucleic acids are connected via phosphate or sulfur-containing linkages. Nucleic acids in some instances comprise DNA, RNA, non-canonical nucleic acids, unnatural nucleic acids, or other nucleoside. In some instances, nucleotides comprise non-canonical bases, sugars, or other moiety. In some instances, nucleotides comprise terminators which are configured to prevent extension reactions. In some instances, such terminators are removed before addition of subsequent nucleotides to the growing chain.

Devices for Molecular Sensing

Provided herein are devices, methods, compositions, and systems useful for molecular sensing. Sensing in some instances comprises detecting the presence or absence of, concentration, and/or identity of a biomolecule or portion of a biomolecule thereof. In some instances, a biomolecule comprises a polymer. In some instances. devices, methods, compositions, and systems are used for sequencing (e.g., nucleic acids). In some instances, solid supports comprise surfaces. Further provided herein are pluralities of devices which are combined to form larger arrays or chips. In some instances, electrical voltages or currents are measured in order to sense molecular changes associated with a charge sensor, molecular sensor, or complex thereof. Arrays of such devices in some instances provide for high-throughput reading of digital information encoded in nucleic acids. In some instances, devices are arrayed on solid supports with each device or groups of devices at an addressable loci. Such larger groups of devices in some instances are present in serve rack units.

The devices provided herein may be used for molecular sensing. Electrodes may be configured to detect changes in voltage, current or resistance to sense biomolecules. In some instances, a device for molecular sensing comprises one or more electrodes. In some instances, a device for molecular sensing comprises one, two, three, four, five or more than five electrodes. In some instances, a device for molecular sensing comprises a source electrode and a drain electrode. In some instances, an electrode of the one or more electrodes is configured as a source. In some instances, an electrode of the one or more electrodes is configured as a drain. In some instances, a device for molecular sensing comprises a passive layer. In some examples, the passive layer comprise an oxide layer. In some examples, the passive layer comprise a nitride layer. In some examples, the passive layer reduces reactivity of the surface to which it is applied. In some instances, a device for molecular sensing comprises a charge sensor binding layer. In some examples, the charge sensor binding layer is a metal, such as gold. In some instances, the one or more electrodes are in electrical communication. In some instances, the electrical communication between the one or more electrodes forms a gap. In some examples, the gap is a nanogap. In some examples, the gap (e.g., nanogap) is spanned by a charge sensor. In some examples, the charge sensor comprises graphene. In some examples, the charge sensor comprises a graphene-enabled field effect transistor (GeFET) or CMOS device. In some instances, a charge sensor is attached to a tether, such as those provided herein. In some instances, the tether is attached to a charge sensor, a molecular sensor, or both. In some examples, the molecular sensor and the charge sensor is in electrical communication. In some examples, the molecular sensor comprises a polymerase. In some examples, the polymerase is an isothermal polymerase, a Phi29 polymerase, or a variant thereof. In some instances, the one or more electrodes are located above one or more base layers. In some examples, the one or more base layers is a passive layer or gate layer.

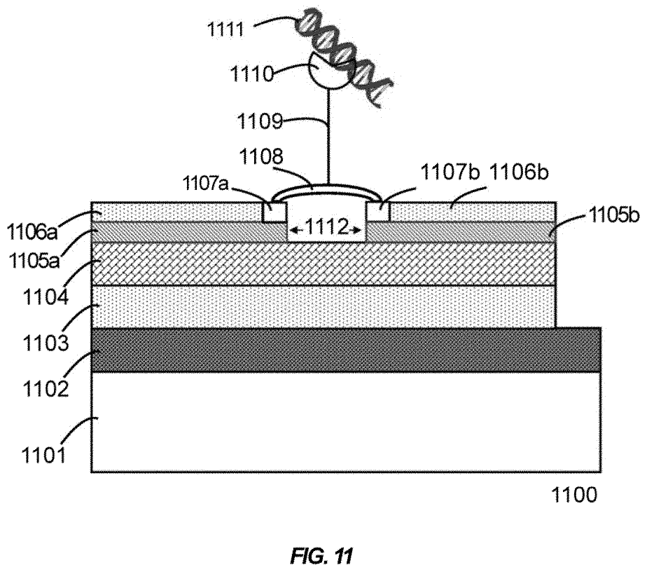

Provided herein are devices for molecular sensing 1100 (FIG. 11). A device in some instances comprises a first electrode 1105a, and a second electrode 1105b. In some instances, a portion of the first electrode 1105a and a portion of the second electrode 1105b are partially covered with a passive layer (1106a and 1106b, respectively). In some instances a portion of the first electrode 1105a and a portion of the second electrode 1105b are partially covered with a charge sensor binding layer (e.g., gold, 1107a and 1107b, respectively). In some instances, the distance between the first charge sensor binding layer 1107a in electrical communication with the first electrode 1105a and the second charge sensor binding layer 1107b in electrical communication with the second electrode 1105b forms a nanogap 1112. In some instances, the nanogap is spanned by a charge sensor 1108. In some instances, the first electrode 1105a is configured as a source. In some instances, the first electrode 1105b is configured as a drain. In some instances, the charge sensor 1108 is attached to a tether 1109. In some instances the tether 1109 is attached to the charge sensor 1108 and a molecular sensor 1110. In some instances, the molecular sensor 1110 is configured to detection an analyte 1111. In some instances, the first electrode 1105a and the second electrode 1105b are located above one or more base layers. In some instances, one or more base layers comprise passive layers or gate layers. In some instances, first electrode 1105a and/or the second electrode 1105b are separated by at least one passive base layer (1104 or 1103). In some instances, one or more base layers (e.g., 1103 and 1101) are separated by a gate layer 1102. In some instances, a gate layer 1102 is located above a base layer 1101. Such devices are in some instances combined into larger arrays. In some instances, devices are integrated into electronics such as a CMOS and connected to a computer.

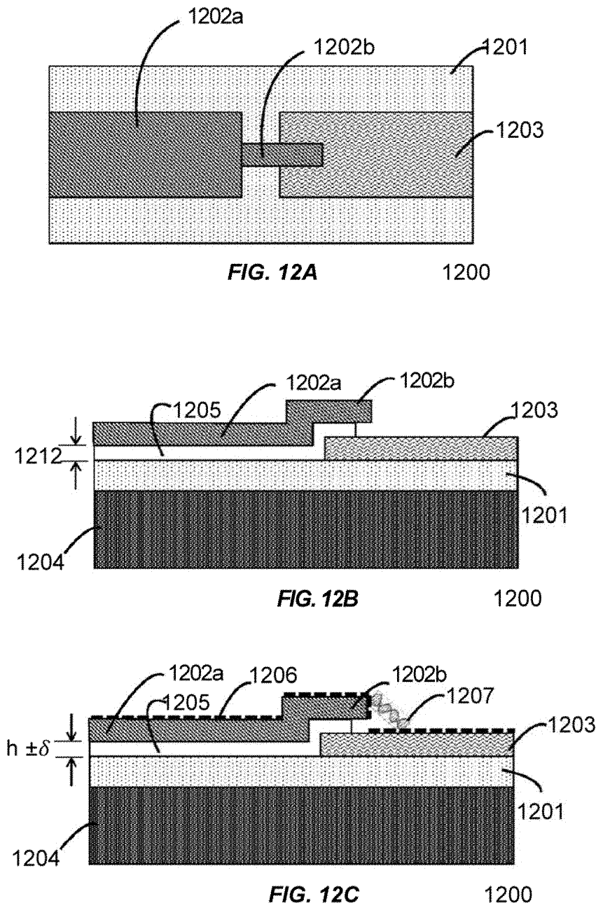

Provided herein is a first device 1200 for molecular sensing (FIGS. 12A-12B). In some instance, the device comprises a first electrode 1202a. In some instances, the first electrode comprises a neck region 1202b. In some instances, the device comprises a second electrode 1203. In some instances, the first electrode 1202a and the second electrode 1202b are located above one or more base layers. In some instances, the first electrode 1202a and the second electrode 1202b are located above a first base layer 1201. In some instances, a first portion of the neck region 1202b overlaps a first portion of the second electrode 1203, such that the first electrode 1202a and the second electrode 1203 are separated by a gap 1212 (e.g., nanogap). In some instances, the nanogap is the smallest defined dimension of a device. In some instances, a second portion of the first electrode 1202a and a second portion of the second electrode 1203 are separated by the passive layer 1205. In some instances, the first base layer 1201 is located above the second base layer 1204, and the first electrode 1202a and the second electrode 1203 are located above the base layers 1201 and 1204. In some instances, a device described herein comprises a coating 1206 (FIG. 12C). In some instances, a coating 1206 is configured to bind a charge sensor 1207. In some instances, a charge sensor 1207 comprises a nanowire 1207. In some instances, a nanowire 1207 comprises a nucleic acid.

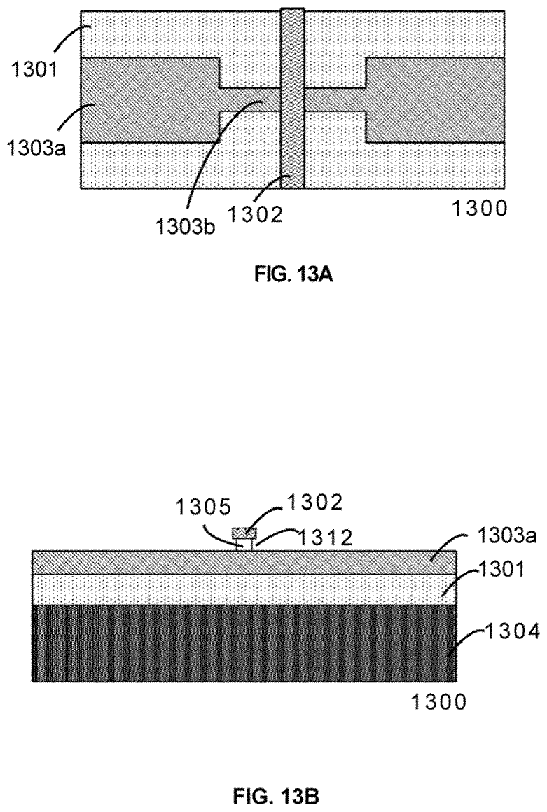

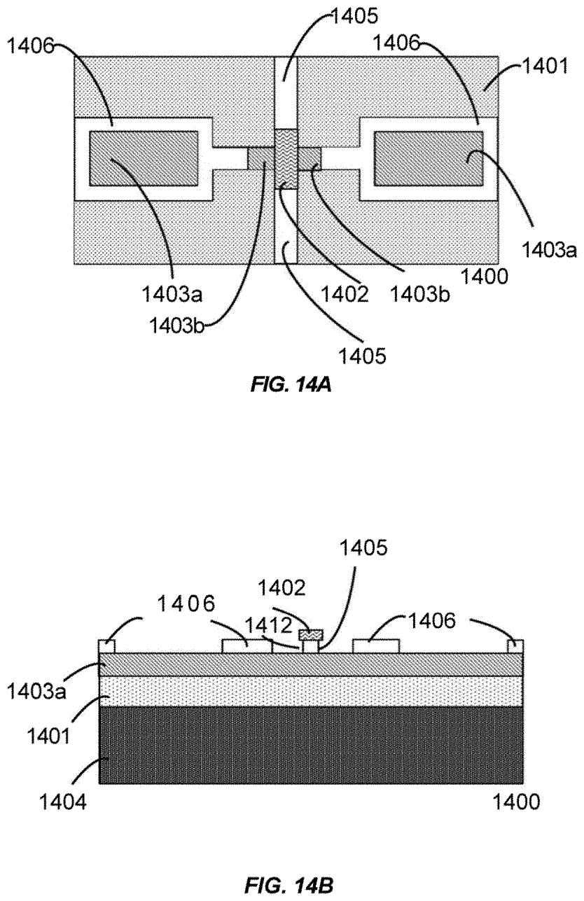

Provided herein is a second device 1300 for molecular sensing (FIGS. 13A-13B). In some instances, the device comprises a first electrode 1302. In some instances, the first electrode is located above the first base layer 1301. In some instances, the device comprises a second electrode 1303a. In some instances, the second electrode 1303a comprises a neck region 1303b. In some instances, a first portion of the neck region 1303b overlaps a first portion of the first electrode 1302, such that the first electrode 1302 and the second electrode 1303a are separated by a nanogap 1312. In some instances, a second portion of the first electrode 1302 and a second portion of the second electrode 1303a are separated by a passive layer 1305. In some instances, a device comprises a first base layer 1301 and a second base layer 1304. In some instances, the first base layer 1301 is located above the second base layer 1302, and the first electrode 1302 and the second electrode 1303a are located above the first base layer 1301. In some instances, the device further comprises a second passive layer 1406. In some instances, the second passive layer 1406 is further configured to overlap with a portion of the second electrode 1403 (FIGS. 14A-14B). In some instances, the second passive layer 1406 passivates the electrode traces.

Provided herein is a third device 1400 for molecular sensing (FIGS. 14A-14B). In some instances, the device comprises a first electrode 1402. In some instances, the first electrode 1402 is located above the first base layer 1401. In some instances, the device comprises a second electrode 1403a, wherein the second electrode 1403a comprises a neck region 1403b. In some instances, a first portion of the neck region 1403b overlaps a first portion of the first electrode 1402, such that the first electrode 1402 and the second electrode 1403a are separated by a nanogap 1412. In some instances, a second portion of the first electrode 1402 and a second portion of the second electrode 1403a are separated by a passive layer 1405. In some instances, a device comprises a first base layer 1401 and a second base layer 1404. In some instances, the first base layer 1401 is located above the second base layer 1402, and the first electrode 1402 and the second electrode 1403a are located above the first base layer 1401. In some instances, the device further comprises a second passive layer 1406.

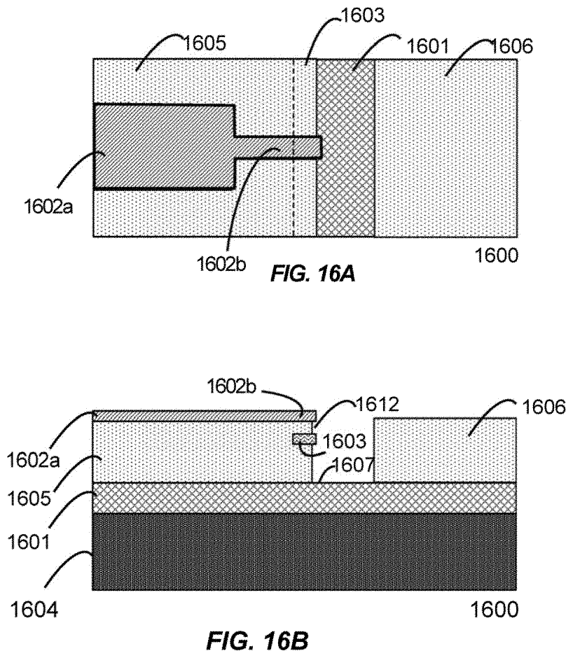

Provided herein is a fourth device 1600 for molecular sensing (FIGS. 16A-16B). In some instances, the device comprises a first electrode 1602a. In some instances, the first electrode 1602a comprises a neck region 1602b. In some instances, the device comprises a passive layer such as 1605 and 1606. In some instances, the passive layer 1605/1606 comprises a channel or well. In some instances, the bottom of the well or channel (or trench) 1607 comprises (or is exposed to) a first base layer 1601. In some instances, the device comprises a second electrode 1603. In some instances, the first electrode 1602a and the second electrode 1603 are located above the first base layer 1601, and the second electrode 1603 is at least partially embedded in the passive layer 1605. In some instances, a first portion of the neck region 1602b overlaps a first portion of the second electrode 1603, such that the first electrode 1602a and the second electrode 1603 are separated by a nanogap 1612. In some instances, a second portion of the first electrode 1602a and a second portion of the second electrode 1603 are separated by the passive layer 1605. In some instances, the device comprises a first base layer 1601 and a second base layer 1604. In some instances, the first base layer 1601 is located above the second base layer 1604, and the first electrode 1602a and the second electrode 1603 are located above the base layers 1601/1604. In some instances, a device stack is etched using the same hard mask so that electrode edges are aligned and a deeper trench (channel or well) is etched below (FIGS. 16A-16B).

In some instances, devices comprise a graphene layer which is configured to detect changes in local currents. In some instances, such layers are coupled to polymerases which provide unique signals (sensing) corresponding to nucleotide incorporation events. In some instances, devices provided herein are used for sequencing nucleic acids. In some instances, devices comprise one or more of a charge sensor and a molecular sensor. In some instances, molecular sensors interact with a biomolecule and convey information about the biomolecule to the charge sensor via changes in current. In some instances, a charge sensor comprises a graphene layer. In some instances, information about the biomolecule comprises information about a monomer of the biomolecule. For example, if the biomolecule is a polynucleotide, the information about the biomolecule comprises information of the nucleotide. In some examples, information about the nucleotide comprises information about the bases (e.g., A, T, C, and G). As another example, if the biomolecule is a peptide, the information about the peptide comprise information about the amino acids.

Provided herein are devices comprising a solid support, a charge sensor, one or more electrodes, and at least one insulating layer. In some instances, the charge sensor comprises a graphene layer. In some instances, the one or more electrodes comprises a gate electrode. In some instances, the one or more electrodes comprises a drain electrode. In some instances, the gate electrode and the drain electrode are in electrical communication via the charge sensor. In some instances, the gate electrode and the drain electrode are in electrical communication via a graphene layer. In some instances, an insulating layer is located between the gate electrode and the drain electrode. In some instances, the solid support comprises a plurality of loci. In some instances, the device further comprises at least one ground shield. In some instances, the device further comprises at least one buried gate (electrode).

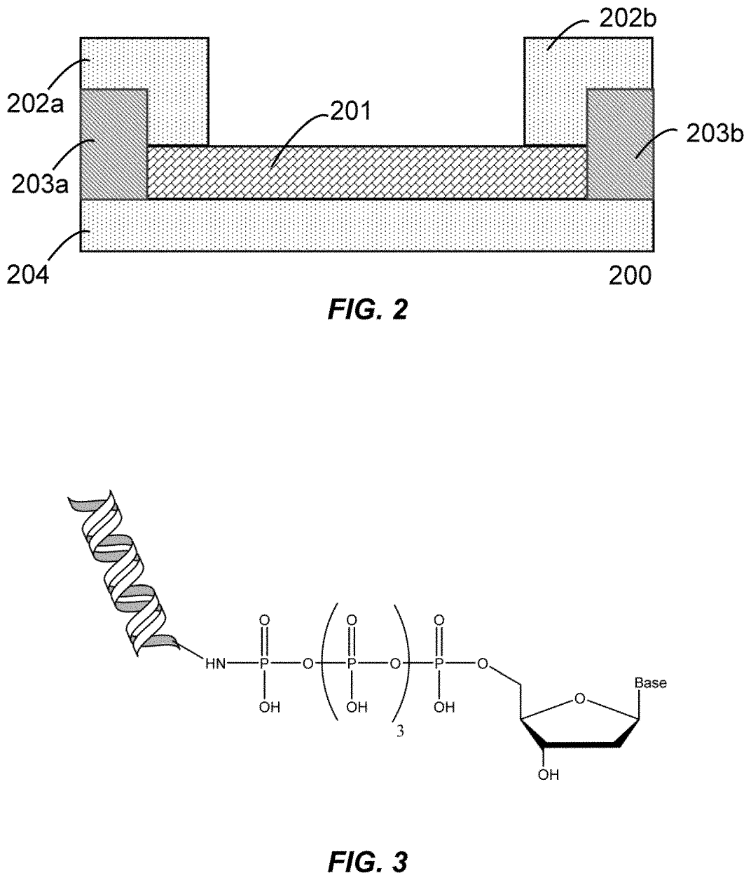

Provided herein is a fifth device 200 as shown in FIG. 2. In some instances the device comprises a solid support 204, a graphene layer 201, a gate electrode 203a, a drain electrode 203b, and at least one insulating layer 202a/202b. In some instances, the insulating layer is located between the gate electrode 203a and the drain electrode 203b. In some instances, the gate electrode 203a and the drain electrode 203b are in electrical communication via the graphene layer 201. In some instances, the gate electrode 203a and the drain electrode 203b comprise platinum. In some instances, the solid support 204 or insulating layer 202a/202b comprises silicon or silicon nitride.

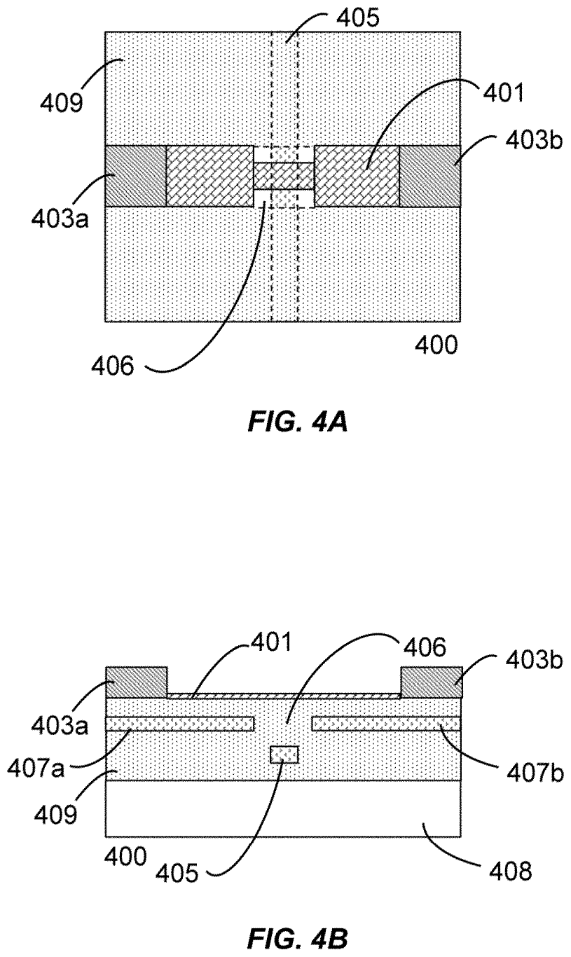

Provided herein is a sixth device 400 as shown in FIGS. 4A-4B. In some instances the device comprises a solid support 408, a graphene layer 401, a gate electrode 403a, a drain electrode 403b, at least one insulating layer 409, at least one ground shield 407a/407b, and a buried gate 405. In some instances, the at least one ground shield 407a/407b comprises an opening which allows electrical contact between the graphene layer 401 and the buried gate 405. In some instances, the insulating layer 409 is located between two or more of the gate electrode 403a, the drain electrode 403b, at least one ground shield 407a/407b, a buried gate 405, and the graphene layer 401. In some instances, the gate electrode 403a and the drain electrode 403b are in electrical communication via the graphene layer 401. In some instances, one or more of the gate electrode 403a, the drain electrode 403b, the buried gate 405, and at least one ground shield 407a/407b comprise platinum. In some instances, the solid support or insulating layer 409 comprises silicon or silicon nitride. In some instances, the buried gate 405 is charged with a voltage to attract or repel molecules from the charge sensor (e.g., graphene layer 401). In some instances, the buried gate 405 is charged with a voltage to modulate currents produced by biomolecules interacting with a molecular sensor described herein.

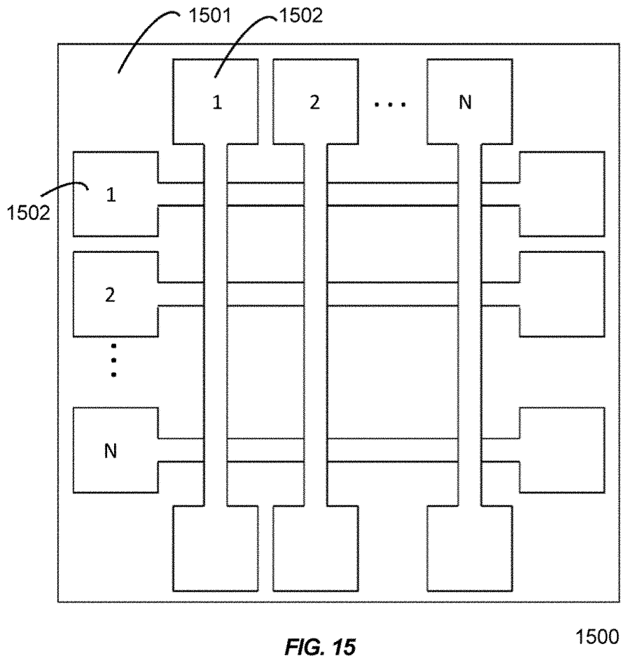

Any devices described above may be arrayed on a solid support. In some instances, devices are arrayed on a solid support such that at least some or a portion of the devices are addressable. In some instances devices 1500 are arrayed on a solid support 1501 in a configuration. In some instances, the first electrodes are placed on the x-axis, and second electrodes 1502 are placed on the y-axis (FIG. 15). Any number of devices are in some instances present in an array. In some instances, arrays comprise 10-1,000,000, 10-100,000, 10-10,000, 10-5,000, 50-1,000,000, 50-100,000, 50-10,000, 50-5,000, 100-1,000,000, 100-100,000, 100-10,000, 100-5,000 or 500-1,00,000 devices. In some instances, arrays comprise at least 5, 10, 20, 50, 100, 200, 500, 1000, 2000, 5000, 10,000, 20,000, 50,000, 100,000, 200,000, or at least 500,000 devices. In some instances, devices are arrayed into larger devices such. In some instances, the density of an array can be from 2 to as many as a billion or more different reaction sites (devices, or loci) per square cm. In some instances, the density of an array is at least 10,000,000 reaction sites/cm², at least 100,000,000; reaction sites/cm², at least 1,000,000,000 reaction sites/cm², at least 2,000,000,000 reaction sites/cm²; at least 100,000 reaction sites/cm²; at least 10,000,000 reaction sites/cm²; at least 100,000 reaction sites/cm²; or at least 10,000 reaction sites/cm². In some instances, the density of the array is 10,000-100,000 reaction sites/cm²; 100,000-500,000 reaction sites/cm²; 100,000-1,000,000 reaction sites/cm²; 10,000-1,000,000 reaction sites/cm²; or 100,000-1,000,000,000 reaction sites/cm². Devices in some instances further comprise vias and/or other connections for electrical communication. In some instances, devices comprise electrodes placed in the z-axis.

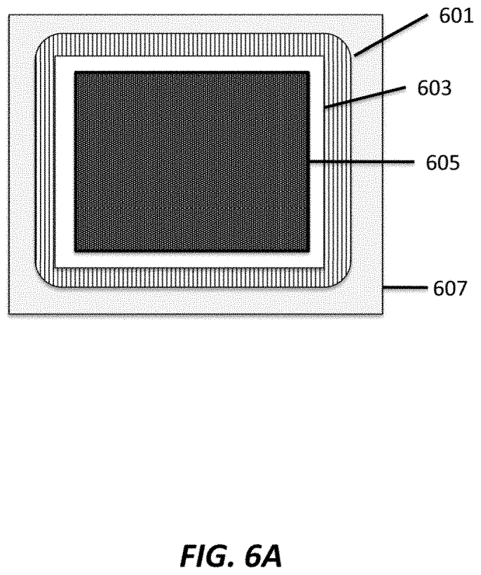

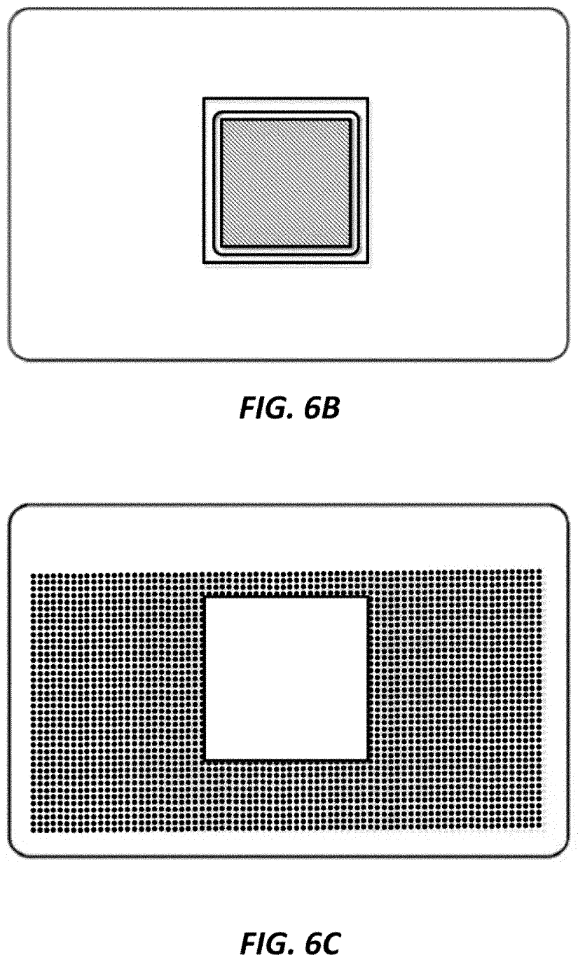

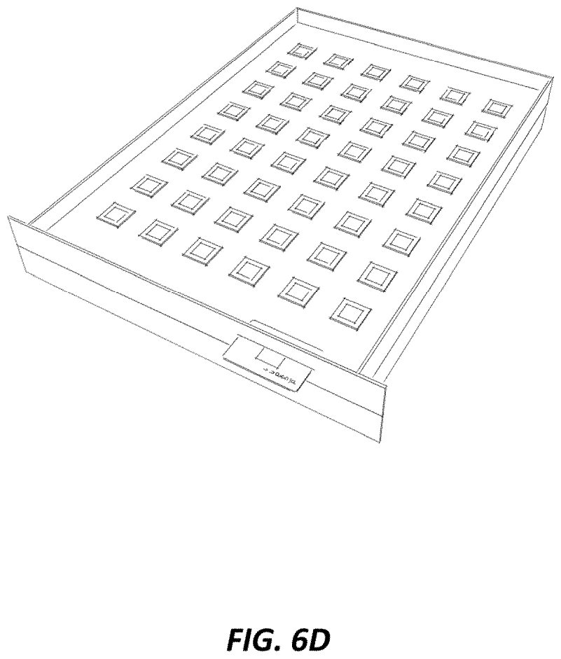

Devices described herein may comprise a solid support. An exemplary solid support can be seen in FIGS. 6B-6C. FIG. 6B shows a front side of the solid support made of glass and comprising a clear window for array and fluidic ports. FIG. 6C shows a back side of the solid support that is a circuit comprising electrical contacts (for example, an LGA 1 mm pitch) and a thermal interface under the solid support area.

Solid supports as described herein comprise an active area. In some instances, the active area comprises addressable solid supports, regions, or loci for molecular sensing. In some instances, the active area comprises addressable regions or loci for nucleic acid storage. In some instances, an active area is in fluid communication with solvents or other reagents. The active area comprises varying dimensions. For example, the dimension of the active area is between about 1 mm to about 50 mm by about 1 mm to about 50 mm. In some instances, the active area comprises a width of at least or about 0.5, 1, 1.5, 2, 2.5, 3, 5, 5, 10, 12, 14, 16, 18, 20, 25, 30, 35, 40, 45, 50, 60, 70, 80, or more than 80 mm. In some instances, the active area comprises a height of at least or about 0.5, 1, 1.5, 2, 2.5, 3, 5, 5, 10, 12, 14, 16, 18, 20, 25, 30, 35, 40, 45, 50, 60, 70, 80, or more than 80 mm. For example, the dimension of the active area is between about 1 μm to about 50 μm by about 1 μm to about 50 μm. In some instances, the active area comprises a width of at least or about 0.5, 1, 1.5, 2, 2.5, 3, 5, 5, 10, 12, 14, 16, 18, 20, 25, 30, 35, 40, 45, 50, 60, 70, 80, or more than 80 μm. In some instances, the active area comprises a height of at least or about 0.5, 1, 1.5, 2, 2.5, 3, 5, 5, 10, 12, 14, 16, 18, 20, 25, 30, 35, 40, 45, 50, 60, 70, 80, or more than 80 μm. In some instances, the active area is 4 to 900, 4 to 500, 4 to 250, 2 to 900, 10 to 900, 25 to 900, 50 to 900, 100 to 900, or 400 to 900 mm². In some instances, the active and any passive area is 4 to 900, 4 to 500, 4 to 250, 2 to 900, 10 to 900, 25 to 900, 50 to 900, 100 to 900, or 400 to 900 mm². In some instances, the active area is 4 to 900, 4 to 500, 4 to 250, 2 to 900, 10 to 900, 25 to 900, 50 to 900, 100 to 900, or 400 to 900 mm². In some instances, the active and any passive area is 4 to 900, 4 to 500, 4 to 250, 2 to 900, 10 to 900, 25 to 900, 50 to 900, 100 to 900, or 400 to 900 mm² for each side of a device described herein. An exemplary active area within a solid support is seen in FIG. 6A. A package 607 comprises an active area 605 within a solid support 603. The package 607 also comprises a fluidics interface 601.

Described herein are devices, compositions, systems and methods for molecular sensors. In some instances, the solid support has a number of sites or positions for molecular sensors. In some instances, the solid support comprises up to or about 10,000 by 10,000 positions in an area. In some instances, the solid support comprises between about 1000 and 20,000 by between about 1000 and 20,000 positions in an area. In some instances, the solid support comprises at least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions by least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions in an area. In some instances, the area is up to 0.25, 0.5, 0.75, 1.0, 1.25, 1.5, or 2.0 inches squared. In some instances, the solid support comprises addressable loci having a pitch of at least or about 0.01, 0.05, 0.1, 0.2, 0.25, 0.3, 0.4, 0.5, 1.0, 1.5, 2.0, 2.5, 3.0, 3.5, 4.0, 4.5, 5, 6, 7, 8, 9, 10, or more than 10 um. In some instances, the solid support comprises addressable loci having a pitch of no more than about 0.01, 0.05, 0.1, 0.2, 0.25, 0.3, 0.4, 0.5, 1.0, 1.5, 2.0, 2.5, 3.0, 3.5, 4.0, 4.5, 5, 6, 7, 8, 9, 10, or no more than 10 um. In some instances, the solid support comprises addressable loci having a pitch of about 5 um. In some instances, the solid support comprises addressable loci having a pitch of about 2 um. In some instances, the solid support comprises addressable loci having a pitch of about 1 um. In some instances, the solid support comprises addressable loci having a pitch of about 0.01 um. In some instances, the solid support comprises addressable loci having a pitch of about 0.02 um. In some instances, the solid support comprises addressable loci having a pitch of about 0.05 um. In some instances, the solid support comprises addressable loci having a pitch of about 0.08 um. In some instances, the solid support comprises addressable loci having a pitch of about 0.1 um. In some instances, the solid support comprises addressable loci having a pitch of about 0.2 um. In some instances, the solid support comprises addressable loci having a pitch of about 0.05 um to about 10 um, about 0.05 to about 1 um, about 0.05 to about 1 um, about 0.1 um to about 1 um, about 0.2 um to about 0.8 um, about 0.3 um to about 0.5 um, about 1 um to about 3 um or about 0.05 um to about 1 um. In some instances, the solid support comprises addressable loci having a pitch of about 0.1 um to about 3 um. In some instances, the solid support comprises addressable loci having a pitch of at least or about 0.01, 0.02, 0.025, 0.03, 0.04, 0.05, 0.1, 0.15, 0.02, 0.25, 0.30, 0.35, 0.4, 0.45, 0.5, 0.6, 0.7, 0.8, 0.9, 1, or more than 1 um. In some instances, the solid support comprises addressable loci having a pitch of about 0.5 um. In some instances, the solid support comprises addressable loci having a pitch of about 0.2 um. In some instances, the solid support comprises addressable loci having a pitch of about 0.1 um. In some instances, the solid support comprises addressable loci having a pitch of about 0.02 um. In some instances, the solid support comprises addressable loci having a pitch of about 0.02 um to about 1 um, about 0.02 to about 0.8 um, about 0.05 to about 0.1 um, about 0.1 um to about 1 um, about 0.2 um to about 0.8 um, about 0.3 um to about 0.5 um, about 0.1 um to about 0.3 um or about 0.05 um to about 0.3 um. In some instances, the solid support comprises addressable loci having a pitch of about 0.01 um to about 0.3 um. In some instances, the solid support comprises addressable loci having a pitch of about 0.05 um to about 1 um.

Devices may comprise gaps or distances between one or more electrodes of various lengths. In some instances, electrodes on each side of the gap are attached to a charge sensor. In some instances, electrodes on each side of the gap are attached to at least one charge sensor. In some instances, electrodes on each side of the gap are attached to at least one shared charge sensor. In some instances, the length of a nanogap is about 5, 10, 15, 20, 25, 30, 35, 40, or about 50 nm. In some instances, the length of a nanogap is about 5-50, 5-25, 5-20, 10-50, 10-30, 15-25, 15-30, 20-40, or 25-50 nm. In some instances, the length of a nanogap is no more than 5, 10, 15, 20, 25, 30, 35, 40, or no more than 50 nm. In some instances, the length of a nanogap is at least 5, 10, 15, 20, 25, 30, 35, 40, or at least 50 nm.

Devices described herein may comprise a neck region. In some instances, an electrode comprises a neck region. In some instances, the neck region is proximal to a nanogap. In some instances, the neck region has width which is at least 5%, 10%, 20%, 30%, 50%, 75% 90%, or more than 95% shorter than the largest dimension of the electrode. In some instances, the neck region has width which is at least 0.5, 1, 1.5, 2, 5, 10, 20, 50, or 100 fold shorter than the largest dimension of the electrode. In some instances, a first electrode comprises a neck region. In some instances, a second electrode comprises a neck region. In some instances, a first electrode comprises a neck region, and a second electrode does not comprise a neck region. In some instances, the neck region has a width of no more than 250, 200, 150, 125, 110, 100, 90, 75, or no more than 50 nm. In some instances, the neck region has a width of about 250, 200, 150, 125, 110, 100, 90, 75, or about 50 nm.

In some instances, each locus of the structure has a width of about 1 um and a distance between the center of each structure of about 2.1 um. In some instances, each locus of the structure has a width of about 0.5 um and a distance between the center of each structure of about 2 um. In some instances, each locus of the structure has a width of about 0.1 um and a distance between the center of each structure of about 0.2 um. Loci may comprise, without limitation, circular, rectangular, tapered, or rounded shapes. Alternatively or in combination, the structures are rigid. In some instances, the rigid structures comprise loci for molecular sensing. In some instances, the rigid structures comprise substantially planar regions, channels, or wells for molecular sensing.

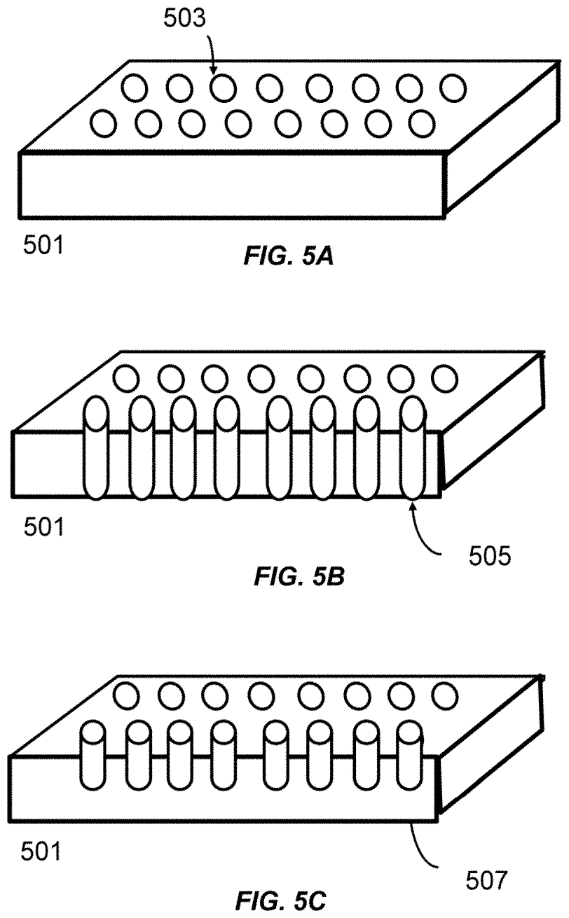

Provided herein are flexible structures having a surface with a plurality of loci for polynucleotide extension. FIGS. 5A-5C show a zoom in of the locus in the flexible structure. Each locus in a portion of the flexible structure 501, may be a substantially planar spot 503 (e.g., flat), a channel 505, or a well 507. Loci may comprise, without limitation, circular, rectangular, tapered, or rounded shapes. Alternatively or in combination, the structures are rigid. In some instances, the rigid structures comprise loci, channels, or wells for polynucleotide synthesis.

In some instances, a well described herein has a width to depth (or height) ratio of 1 to 0.01. In some instances, the width is a measurement of the width at the narrowest segment of the well. In some instances, a well described herein has a width to depth (or height) ratio of 0.5 to 0.01. In some instances, the width is a measurement of the width at the narrowest segment of the well. In some instances, a well described herein has a width to depth (or height) ratio of about 0.01, 0.05, 0.1, 0.15, 0.16, 0.2, 0.5, or 1. Provided herein are structures for molecular sensing comprising a plurality of discrete loci for molecular sensing. Exemplary structures for the loci include, without limitation, substantially planar regions, channels, wells or protrusions. Structures described herein may comprise a plurality of clusters, each cluster comprising a plurality of wells, loci or channels. Alternatively, described herein may comprise a homogeneous arrangement of wells, loci or channels. Structures provided herein may comprise wells having a height or depth from about 0.1 um to about 5 um, from about 0.1 um to about 400 um, from about 0.1 um to about 300 um, from about 0.1 um to about 200 um, from about 0.1 um to about 100 um, from about 0.1 um to about 0.5 um, or from about 0.01 um to about 0.5 um. In some instances, the height of a well is less than 0.10 um, less than 0.08 um, less than 0.6 um, less than 0.40 um or less than 0.2 um. In some instances, well height is about 10, 20, 30, 40, 50, 60, 70, 80, 90, 100, 200, 300, 400, 500 nm or more. In some instances, the height or depth of the well is at least 10, 25, 50, 75, 100, 200, 300, 400, 500, 600, 700, 800, 900, 1000, or more than 1000 nm. In some instances, the height or depth of the well is in a range of about 10 nm to about 1000 nm, about 25 nm to about 900 nm, about 50 nm to about 800 nm, about 75 nm to about 700 nm, about 100 nm to about 600 nm, or about 200 nm to about 500. In some instances, the height or depth of the well is in a range of about 50 nm to about 1 um. In some instances, well height is about 10, 20, 30, 40, 50, 60, 70, 80, 90, 100, 200, 300, 400, 500, 700, 800, 900 or about 1000 nm.

Structures for molecular sensing provided herein may comprise channels. The channels may have a width to depth (or height) ratio of 1 to 0.01. In some instances, the width is a measurement of the width at the narrowest segment of the microchannel. In some instances, a channel described herein has a width to depth (or height) ratio of 0.5 to 0.01. In some instances, the width is a measurement of the width at the narrowest segment of the microchannel. In some instances, a channel described herein has a width to depth (or height) ratio of about 0.01, 0.05, 0.1, 0.15, 0.16, 0.2, 0.5, or 1.

Described herein are structures for molecular sensing comprising a plurality of discrete loci. Structures comprise, without limitation, substantially planar regions, channels, protrusions, or wells for molecular sensing. In some instances, structures described herein are provided comprising a plurality of channels, wherein the height or depth of the channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some cases, the height of a channel is less than 100 um, less than 80 um, less than 60 um, less than 40 um or less than 20 um. In some cases, channel height is about 10, 20, 30, 40, 50, 60, 70, 80, 90, 100, 200, 300, 400, 500 um or more. In some instances, the height or depth of the channel is at least 10, 25, 50, 75, 100, 200, 300, 400, 500, 600, 700, 800, 900, 1000, or more than 1000 nm. In some instances, the height or depth of the channel is in a range of about 10 nm to about 1000 nm, about 25 nm to about 900 nm, about 50 nm to about 800 nm, about 75 nm to about 700 nm, about 100 nm to about 600 nm, or about 200 nm to about 500. Channels described herein may be arranged on a surface in clusters or as a homogeneous field.

The width of a locus on the surface of a structure for molecular sensing described herein may be from about 0.1 um to about 500 um, from about 0.5 um to about 500 um, from about 1 um to about 200 um, from about 1 um to about 100 um, from about 5 um to about 100 um, or from about 0.1 um to about 100 um, for example, about 90 um, 80 um, 70 um, 60 um, 50 um, 40 um, 30 um, 20 um, 10 um, 5 um, 1 um or 0.5 um. In some instances, the width of a locus is less than about 100 um, 90 um, 80 um, 70 um, 60 um, 50 um, 40 um, 30 um, 20 um or 10 um. In some instances, the width of a locus is at least 10, 25, 50, 75, 100, 200, 300, 400, 500, 600, 700, 800, 900, 1000, or more than 1000 nm. In some instances, the width of a locus is in a range of about 10 nm to about 1000 nm, about 25 nm to about 900 nm, about 50 nm to about 800 nm, about 75 nm to about 700 nm, about 100 nm to about 600 nm, or about 200 nm to about 500. In some instances, the width of a locus is in a range of about 50 nm to about 1000 nm. In some instances, the distance between the center of two adjacent loci is from about 0.1 um to about 500 um, 0.5 um to about 500 um, from about 1 um to about 200 um, from about 1 um to about 100 um, from about 5 um to about 200 um, from about 5 um to about 100 um, from about 5 um to about 50 um, or from about 5 um to about 30 um, for example, about 20 um. In some instances, the total width of a locus is about 5 um, 10 um, 20 um, 30 um, 40 um, 50 um, 60 um, 70 um, 80 um, 90 um, or 100 um. In some instances, the total width of a locus is about 1 um to 100 um, 30 um to 100 um, or 50 um to 70 um. In some instances, the distance between the center of two adjacent loci is from about 0.5 um to about 2 um, 0.5 um to about 2 um, from about 0.75 um to about 2 um, from about 1 um to about 2 um, from about 0.2 um to about 1 um, from about 0.5 um to about 1.5 um, from about 0.5 um to about 0.8 um, or from about 0.5 um to about 1 um, for example, about 1 um. In some instances, the total width of a locus is about 50 nm, 0.1 um, 0.2 um, 0.3 um, 0.4 um, 0.5 um, 0.6 um, 0.7 um, 0.8 um, 0.9 um, 1 um, 1.1 um, 1.2 um, 1.3 um, 1.4 um, or 1.5 um. In some instances, the total width of a locus is about 0.5 um to 2 um, 0.75 um to 1 um, or 0.9 um to 2 um.

In some instances, each locus supports the sensing of a population of polynucleotides having a different sequence than a population of polynucleotides grown on another locus. Provided herein are surfaces which comprise at least 10, 100, 256, 500, 1000, 2000, 3000, 4000, 5000, 6000, 7000, 8000, 9000, 10000, 11000, 12000, 13000, 14000, 15000, 20000, 30000, 40000, 50000 or more clusters. Provided herein are surfaces which comprise more than 2,000; 5,000; 10,000; 20,000; 30,000; 50,000; 100,000; 200,000; 300,000; 400,000; 500,000; 600,000; 700,000; 800,000; 900,000; 1,000,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150, 200, 500 or more loci. In some cases, each cluster includes 50 to 500, 50 to 200, 50 to 150, or 100 to 150 loci. In some cases, each cluster includes 100 to 150 loci. In some instances, each cluster includes 109, 121, 130 or 137 loci.

Provided herein are loci having a width at the longest segment of 5 to 100 um. In some cases, the loci have a width at the longest segment of about 30, 35, 40, 45, 50, 55 or 60 um. In some cases, the loci are channels having multiple segments. In some instances, each segment has a center to center distance apart of 5 to 50 um. In some cases, the center to center distance apart for each segment is about 5, 10, 15, 20 or 25 um.

Provided herein are loci having a width at the longest segment of 5 to 500 nm. In some cases, the loci have a width at the longest segment of about 30, 35, 40, 45, 50, 55, 60, 80, or 100 nm. In some cases, the loci are channels having multiple segments. In some instances, each segment has a center to center distance apart of 5 to 50 nm. In some cases, the center to center distance apart for each segment is about 5, 10, 15, 20, 25, 50, 100, or 200 nm.

In some instances, the number of distinct polynucleotides synthesized on the surface of a structure described herein is dependent on the number of distinct loci available in the substrate. In some instances, the density of loci within a cluster of a substrate is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm², 10⁴ loci per mm², 10⁵ loci per mm², 10⁶ loci per mm², or more. In some cases, a substrate comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some cases, a substrate comprises from about 10⁴ loci per mm² to about 10⁵ mm². In some cases, a substrate comprises from about 10⁵ loci per mm² to about 10⁷ mm². In some cases, a substrate comprises at least 10⁵ loci per mm². In some cases, a substrate comprises at least 10⁶ loci per mm². In some cases, a substrate comprises at least 10⁷ loci per mm². In some cases, a substrate comprises from about 10⁴ loci per mm² to about 10⁵ mm². In some instances, the density of loci within a cluster of a substrate is at least or about 1 locus per um², 10 loci per um², 25 loci per um², 50 loci per um², 65 loci per um², 75 loci per um², 100 loci per um², 130 loci per um², 150 loci per um², 175 loci per um², 200 loci per um², 300 loci per um², 400 loci per um², 500 loci per um², 1,000 loci per um² or more. In some cases, a substrate comprises from about 10 loci per um² to about 500 um², from about 25 loci per um² to about 400 um², from about 50 loci per um² to about 500 um², from about 100 loci per um² to about 500 um², from about 150 loci per um² to about 500 um², from about 10 loci per um² to about 250 um², from about 50 loci per um² to about 250 um², from about 10 loci per um² to about 200 um², or from about 50 loci per um² to about 200 um².

In some instances, the distance between the centers of two adjacent loci within a cluster is from about 10 um to about 500 um, from about 10 um to about 200 um, or from about 10 um to about 100 um. In some cases, the distance between two centers of adjacent loci is greater than about 10 um, 20 um, 30 um, 40 um, 50 um, 60 um, 70 um, 80 um, 90 um or 100 um. In some cases, the distance between the centers of two adjacent loci is less than about 200 um, 150 um, 100 um, 80 um, 70 um, 60 um, 50 um, 40 um, 30 um, 20 um or 10 um. In some cases, the distance between the centers of two adjacent loci is less than about 10000 nm, 8000 nm, 6000 nm, 4000 nm, 2000 nm 1000 nm, 800 nm, 600 nm, 400 nm, 200 nm, 150 nm, 100 nm, 80 um, 70 nm, 60 nm, 50 nm, 40 nm, 30 nm, 20 nm or 10 nm. In some instances, each square meter of a structure described herein allows for at least 10⁷, 10⁸, 10⁹, 10¹⁰, 10¹¹ loci. In some instances, each locus supports one polynucleotide. In some instances, 10⁹ polynucleotides are supported on less than about 6, 5, 4, 3, 2 or 1 m² of a structure described herein.

In some instances, a structure described herein provides support for the sensing of more than 2,000; 5,000; 10,000; 20,000; 30,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the structure provides support for the sensing of more than 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the structure provides a surface environment for the growth of polynucleotides having at least 50, 60, 70, 75, 80, 85, 90, 95, 100, 110, 120, 130, 140, 150, 160, 175, 200, 225, 250, 275, 300, 325, 350, 375, 400, 425, 450, 475, 500 bases or more. In some arrangements, structures for molecular sensing described herein comprise sites for molecular sensing in a uniform arrangement.

In some instances, polynucleotides are synthesized on distinct loci of a structure. In some instances, each locus supports the sensing of a population of polynucleotides. In some cases, each locus supports the sensing of a population of polynucleotides having a different sequence than a population of polynucleotides sensed on another locus. In some instances, the loci of a structure are located within a plurality of clusters. In some instances, a structure comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a structure comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150 or more loci. In some instances, each cluster includes 50 to 500, 100 to 150, or 100 to 200 loci. In some instances, each cluster includes 109, 121, 130 or 137 loci. In some instances, each cluster includes 5, 6, 7, 8, 9, 10, 11 or 12 loci. In some instances, polynucleotides from distinct loci within one cluster have sequences that, when assembled, encode for a contiguous longer polynucleotide of a predetermined sequence. In some instances, each of the polynucleotides comprise a plurality of different nucleotide bases (e.g., A, T, C, G, etc.).

In some instances, a structure described herein is about the size of a plate (e.g., chip), for example between about 40 and 120 mm by between about 25 and 100 mm. In some instances, a structure described herein has a diameter less than or equal to about 1000 mm, 500 mm, 450 mm, 400 mm, 300 mm, 250 nm, 200 mm, 150 mm, 100 mm or 50 mm. In some instances, the diameter of a substrate is between about 25 mm and 1000 mm, between about 25 mm and about 800 mm, between about 25 mm and about 600 mm, between about 25 mm and about 500 mm, between about 25 mm and about 400 mm, between about 25 mm and about 300 mm, or between about 25 mm and about 200. Non-limiting examples of substrate size include about 300 mm, 200 mm, 150 mm, 130 mm, 100 mm, 84 mm, 76 mm, 54 mm, 51 mm and 25 mm. In some instances, a substrate has a planar surface area of at least 100 mm²; 200 mm²; 500 mm²; 1,000 mm²; 2,000 mm²; 4,500 mm²; 5,000 mm²; 10,000 mm²; 12,000 mm²; 15,000 mm²; 20,000 mm²; 30,000 mm²; 40,000 mm²; 50,000 mm² or more. In some instances, the thickness is between about 50 mm and about 2000 mm, between about 50 mm and about 1000 mm, between about 100 mm and about 1000 mm, between about 200 mm and about 1000 mm, or between about 250 mm and about 1000 mm. Non-limiting examples thickness include 275 mm, 375 mm, 525 mm, 625 mm, 675 mm, 725 mm, 775 mm and 925 mm. In some instances, the thickness is at least or about 0.5 mm, 1.0 mm, 1.5 mm, 2.0 mm, 2.5 mm, 3.0 mm, 3.5 mm, 4.0 mm, or more than 4.0 mm. In some cases, the thickness of varies with diameter and depends on the composition of the substrate. For example, a structure comprising materials other than silicon may have a different thickness than a silicon structure of the same diameter. Structure thickness may be determined by the mechanical strength of the material used and the structure must be thick enough to support its own weight without cracking during handling.

Described herein are devices where two or more solid supports are assembled. In some instances, solid supports are interfaced together on a larger unit. Interfacing may comprise exchange of fluids, electrical signals, or other medium of exchange between solid supports. This unit may be capable of interfacing with any number of servers, computers, or networked devices. For example, a plurality of solid support is integrated onto a rack unit or mounted onto a rack unit, which can be conveniently inserted or removed from a server rack. The rack unit may comprise any number of solid supports. In some instances the rack unit comprises about 1, 2, 5, 10, 20, 50, 100, 200, 500, 1000, 2000, 5000, 10,000, 20,000, 50,000, 100,000 or 100,000 solid supports. In some instances the rack unit comprises at least 1, 2, 5, 10, 20, 50, 100, 200, 500, 1000, 2000, 5000, 10,000, 20,000, 50,000, 100,000 or more than 100,000 solid supports. In some instances, the rack unit comprises at most 1, 2, 5, 10, 20, 50, 100, 200, 500, 1000, 2000, 5000, 10,000, 20,000, 50,000, 100,000 or 100,000 solid supports. In some instances, all or a portion of the solid supports of a rack unit are in fluidic communication, electronic communication, or both. In some instances, the server rack comprises about 10, 20, 50, 80, 100, 200, 500, 800, or 1000 rack units. In some instances, the server rack comprises at least about 10, 20, 50, 80, 100, 200, 500, 800, or 1000 rack units. In some instances, the server rack comprises at most about 10, 20, 50, 80, 100, 200, 500, 800, or 1000 rack units. In some instances, all or a portion of the rack units of a rack server are in fluidic communication, electronic communication, or both. In some instances, two or more solid supports are not interfaced with each other. In some instances, two or more rack units comprising solid supports, such as those described herein, are stacked vertically. Fluidic communication, electronic communication, or both may be formed using, by way of non-limiting example, one or more tubes (e.g., microfluidic tubes), valves, actuators, robotics, etc.

Nucleic acids (and the information stored in them) present on solid supports can be accessed from the rack unit. See e.g., FIG. 6D. Access includes removal of polynucleotides from solid supports, direct analysis of polynucleotides on the solid support, or any other method which allows the information stored in the nucleic acids to be manipulated or identified. Information in some instances is accessed from a plurality of racks, a single rack, a single solid support in a rack, a portion of the solid support, or a single locus on a solid support. In various instances, access comprises interfacing nucleic acids with additional devices such as mass spectrometers, HPLC, sequencing instruments, PCR thermocyclers, or other device for manipulating nucleic acids. Access to nucleic acid information in some instances is achieved by cleavage of polynucleotides from all or a portion of a solid support.

In some instances, the rack unit or rack server is located in a data center. In some instances, the data center employs mechanical structures used for mounting conventional computing and data storage resources in rack units, for example, openings adapted to support disk drives, processing blades, or other computer equipment. In some instances, computer systems, such as those provided herein, are used to retrieve polynucleotides from one or more rack units on one or more rack servers. In some instances, a user (e.g., technician, researcher, customer, etc.), computer system, or both directs retrieval of one or more rack units on one or more rack servers. In some instances, a rack unit can be retrieved from a rack server using a robotic system, such as a robotic arm. In some instances, the robotic system is in communication with the computer system. The robotic system may be used to interface any component of a data storage system with another component of the data storage system. In some instances, interfacing comprises transferring, storing, moving, processing, or retrieving. In some instances, the robotic system moves a solid support between components (e.g., units or chambers) of the data storage system. A component may comprise, by way of non-limiting example, synthesis unit, storage unit, amplification unit, etc. Rack units or servers in some instances comprise units for inflow of reagents or outflow of waste or synthesis products.

Cleavage in some instances comprises exposure to chemical reagents (ammonia or other reagent), electrical potential, radiation, heat, light, acoustics, or other form of energy capable of manipulating chemical bonds. In some instances, cleavage occurs by charging one or more electrodes in the vicinity of the polynucleotides. In some instances, electromagnetic radiation in the form of UV light is used for cleavage of polynucleotides. In some instances, a lamp is used for cleavage of polynucleotides, and a mask mediates exposure locations of the UV light to the surface. In some instances, a laser is used for cleavage of polynucleotides, and a shutter opened/closed state controls exposure of the UV light to the surface. In some instances, a computer system, such as those provided herein, directs the opened/closed state of the shutter. In some instances, access to nucleic acid information (including removal/addition of racks, solid supports, reagents, nucleic acids, or other component) is completely automated (e.g., using computer systems provided herein). In some instances, chips have one or more contacts. In some instances, chips comprise at least 5, 10, 15, 20, 25, 30, 35, 40, 45, 50, 55, 60, 75, 100, or more than 200 contacts.

Devices herein may comprise biomolecules attached to one or more electrodes. In some instances, the biomolecule is a charge sensor. In some instances, the charge sensor bridges or spans at least two electrodes. In some instances, the charge sensor comprises a nanowire. In some instances, the charge sensor comprises a polymer. In some instances, the polymer comprises nucleic acids. In some instances, the charge sensor is configured to bind to one or more electrodes of the devices described herein. In some instances, a charge sensor comprises a detection device that translates perturbations at its surface or in its surrounding electrical field into an electrical signal. For example, a charge sensor can translate the arrival or departure of a reaction component into an electrical signal. A charge sensor can also translate interactions between two reaction components, or conformational changes in a single reaction component, into an electrical signal. In some instances, a charge sensor comprises a nanowire. In some instances, a charge sensor comprises a GeFET. An exemplary charge sensor is a field effect transistor (FET) such as a single-walled carbon nanotube (SWNT) based FET, silicon nanowire (SiNW) FET, graphene nanoribbon FET (and related nanoribbon FETs fabricated from 2D materials such as MoS₂, silicene, or other material), tunnel FET (TFET), and steep subthreshold slope devices. In some instances, nanowires comprise a polymer. In some instances, the polymer comprises at least one nucleic acid, amino acid, sugar, or lipid. In some instances, the charge sensor comprises carbon. In some instances the charge sensor is attached to at least one of the first electrode or the second electrode via sulfur-gold interaction. In some instances, the charge sensor is further attached to a molecular sensor via a tether, such as those described herein. In some instances, the charge sensor comprises nucleic acids. In some instances the charge sensor comprises DNA or RNA. In some instances, the charge sensor comprises 20-500, 50-500, 100-500, 150-1000, 150-500, 250-1000, 500-1000, or 600-1000 nucleic acids. In some instances, the charge sensor comprises no more than 1000, 750, 500, 250, 200, 100, 50, or no more than 20 nucleic acids. In some instances, the charge sensor comprises about 1000, 750, 500, 250, 200, 100, 50, or about 20 nucleic acids. In some instances, the charge sensor comprises at least 1000, 750, 500, 250, 200, 100, 50, or at least 20 nucleic acids. In some instances, at least one nucleic acid is attached to a tether. In some instances, the polymer comprises a moiety for attachment to one or more electrodes. In some instances, the charge sensor is attached to at least one of the first electrode or the second electrode via sulfur-gold interaction. In some instances, then length of the charge sensor is about 10, 15, 20, 25, 30, 35, or about 40 nm. In some instances, then length of the charge sensor is no more than 10, 15, 20, 25, 30, 35, or no more than 40 nm. In some instances, at least 1, 2, 5, 10, 15, 20, 25, 50, 75, or 80% of the electrodes are attached to a molecular sensor.

Devices herein may comprise molecular sensors. In some instances, a molecular sensor is configured to detect the presence or absence of an analyte. In some instances, a molecular sensor is in electrical communication with a charge sensor, optionally through a tether. In some instances, the molecular sensor comprises a polymerase. Any of a variety of polymerases are in some instances used in a method or composition including protein-based enzymes isolated from biological systems and functional variants thereof. In some instances, the polymerase is configured to catalyze the polymerization of a nucleic acid strand using an existing nucleic acid as a template. Polymerases include but are not limited to DNA polymerases and RNA polymerases. Exemplary DNA polymerases include those that have been classified by structural homology into families identified as A, B, C, D, X, Y, and RT. DNA Polymerases in Family A include T7 DNA polymerase, eukaryotic mitochondrial DNA Polymerase gamma, E. coli DNA Pol I, Thermus aquaticus Pol I, and Bacillus stearothermophilus Pol I. DNA Polymerases in Family B include, eukaryotic DNA polymerases alpha, delta, and epsilon; DNA polymerase ζ; T4 DNA polymerase, Phi29 DNA polymerase, and RB69 bacteriophage DNA polymerase. Family C includes the E. coli DNA Polymerase III alpha subunit. Family D includes polymerases derived from the Euryarchaeota subdomain of Archaea. DNA Polymerases in Family X include eukaryotic polymerases Pol beta, pol sigma, Pol X, and Pol p. and S. cerevisiae Pol4. DNA polymerases in Family Y include Pol eta, Pol iota, Pol kappa, E. coli Pol IV (DINB) and E. coli Pol V (UmuD′2C). The RT (reverse transcriptase) family of DNA polymerases includes retrovirus reverse transcriptases and eukaryotic telomerases. Exemplary RNA polymerases include but are not limited to viral RNA polymerases such as T7 RNA polymerase; Eukaryotic RNA polymerases such as RNA polymerase I, RNA polymerase II, RNA polymerase III, RNA polymerase IV, and RNA polymerase V; and Archaea RNA polymerase.

A molecular sensor may be attached to a charge sensor via a tether (or linkage). In some instances, a molecular sensor is attached to a charge sensor by a non-covalent linkage such as one formed between a receptor and a ligand. Linkages include but are not limited to those between streptavidin (or variants or analogs thereof) and biotin (or its analogs), those between complementary nucleic acids, those between antibodies and epitopes and the like. In some instances, a conducting tether is used to attach a molecular sensor to a charge sensor. Exemplary conducting tethers include those having a structure that includes doped polythiophene, poly(3,4-ethylenedioxythiophene), polyacetylenes, polypyrroles, polyanilines, polyfluorenes, polyphenylenes, polypyrenes, polyazulenes, polynaphthalenes, polycarbazoles, polyindoles, or polyazepines. Charge doping of tether structures I some instances is achieved by oxidation of the polymer. Exemplary conducting tethers and methods for their creation are set forth in Vemitskaya et al. Russ. Chem. Rev. 66:44311 (1997); MacDiarmid, Angew. Chem., mt. Ed. 40:2581-2590 (2001); or McNeill et al., Aust. J. Chem. 16:1056-75 (1963). In some instances, the molecular sensor is a polymerase. In some instances, the charge sensor is a nanowire. In some instances, In some instances, a molecular sensor is attached to a charge sensor via a noncovalent interaction, such as pi bonding, electrostatics, F—S interaction, or other noncovalent binding modality. In some instances, molecular sensor is attached to a charge sensor via a linker using a conjugation. In some instances, the conjugation comprises nucleophile/carbonyl; an azide/phosphine; 1,4 Michael addition, 1,3-dipolar cycloaddition, inverse electron demand cycloaddition; olefin metathesis; or cross-coupling reaction. In some instances, a ternary complex is formed among a molecular sensor, a biomolecule, and a primer. In some instances, a ternary complex is attached to the charge sensor via a primer. In some instances, a ternary complex is attached to the charge sensor via a molecular sensor. In some instances, a ternary complex is attached to the charge sensor via a biomolecule.

A molecular sensor may comprise a graphene-binding moiety configured to attach the molecular sensor to a charge sensor comprising a graphene layer. Such moieties may bind to other pi-based charge sensors such as a graphene layer. In some instances, graphene layers are 1-5 atoms thick. In some instances, graphene layers are about 1 atom thick. In some instances, the graphene binder comprises an aromatic group. In some instances, the graphene binder comprises an aryl or heteroaryl group. In some instances, the graphene binder comprises a C₆-C₃₀ aryl or heteroaryl group. In some instances, the graphene binder comprises a C₆-C₂₀ aryl or heteroaryl group. In some instances, the graphene binder comprises a C₆-C₁₅ aryl or heteroaryl group. In some instances, the graphene binder comprises a C₆-C₁₀ aryl or heteroaryl group. In some instances, the graphene binder comprises a C₁₀-C₃₀ aryl or heteroaryl group. In some instances, the graphene binder comprises a C₁₅-C₃₀ aryl or heteroaryl group. In some instances, the graphene binder comprises an aromatic hydrocarbon. In some instances, the graphene binder comprises naphthalene, biphenyl, fluorene, anthracene, phenanthrene, tetracene, chrysene, triphenylene, pyrene, pentacene, perylene, benzo[a]pyrene, corannulene, benzo[ghi]perylene, coronene, ovalene, or benzo[c]fluorene. In some instances, a ternary complex (comprising a molecular sensor, biomolecule, and a primer) is attached to the graphene binder via the primer, the polymerase, or the biomolecule.

Fabrication of Nanoelectric Devices

Provided herein are methods of fabricating the devices and surfaces for molecular sensing. In some instances, methods herein comprise deposition materials onto one or more base layers. In some instances, methods comprise depositing materials to generate electrodes or passive layers. In some instances, methods comprise patterning electrodes or passive layers. In some instances, methods described herein comprise etching. In some instances, methods described herein comprise isotropic or substantially isotropic etching. In some instances, etching generates edges of an electrode that are undercut. In some instances, a method comprises at least some of the steps a) providing one or more base layers; b) depositing material to generate a second electrode; c) patterning the second electrode; d) optionally planarizing; e) depositing material to generate a passive layer; f) depositing material to generate the first electrode; g) patterning the first electrode; and h) isotropically etching the passive layer, such that the edges of the first electrode are undercut. In some instances, methods further comprise depositing a material configured to bind to a nanowire. In some instances, methods further comprise depositing gold on the top layer of the device. In some instances, methods further comprise depositing gold on one or more electrodes. In some instances the method comprises etching or lithography. In some instances, the method comprises RIE (reactive ion etching). In some instances, patterning comprises lithography and/or RIE. In some instances, the method does not comprise e-beam or DUV (deep ultraviolet light) lithography. In some instances, the method comprises deposition of gold on the first electrode and the second electrode.

Provided herein are methods to support the immobilization of a biomolecule (such as a nanowire) on a substrate. In some instances, a surface of a structure described herein comprises a material and/or is coated with a material that facilitates a coupling reaction with the biomolecule for attachment. To prepare a structure for biomolecule immobilization, surface modifications may be employed that chemically and/or physically alter the substrate surface by an additive or subtractive process to change one or more chemical and/or physical properties of a substrate surface or a selected site or region of the surface. For example, surface modification involves (1) changing the wetting properties of a surface, (2) functionalizing a surface, e.g., providing, modifying or substituting surface functional groups, (3) defunctionalizing a surface, e.g., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface. In some instances, the surface of a structure is selectively functionalized to produce two or more distinct areas on a structure, wherein at least one area has a different surface or chemical property that another area of the same structure. Such properties include, without limitation, surface energy, chemical termination, surface concentration of a chemical moiety, and the like.

The surfaces provided herein can have an active area, a passive area, or both. The active area may referred to as an actively functionalized surface. In some instances, the active area is functionalized with an active material. In some instances, the passive area is functionalized with a passive material. In some instances, a surface of a structure disclosed herein is modified to comprise one or more actively functionalized surfaces configured to bind to both the surface of the substrate and a biomolecule, thereby supporting a coupling reaction to the surface. In some instances, the surface is also functionalized with a passive material that does not efficiently bind the biomolecule. In some examples, the surface functionalized with a passive material prevents biomolecule attachment at sites where the passive functionalization agent is bound. In some cases, the surface comprises an active layer only defining distinct loci for biomolecule support.

In some instances, functionalization comprises deposition of a functionalization agent to a structure by any deposition technique, including, but not limiting to, chemical vapor deposition (CVD), atomic layer deposition (ALD), plasma enhanced CVD (PECVD), plasma enhanced ALD (PEALD), metal organic CVD (MOCVD), hot wire CVD (HWCVD), initiated CVD (iCVD), modified CVD (MCVD), vapor axial deposition (VAD), outside vapor deposition (OVD), physical vapor deposition (e.g., sputter deposition, evaporative deposition), and molecular layer deposition (MLD).

Any step or component in the following functionalization process be omitted or changed in accordance with properties desired of the final functionalized substrate. In some cases, additional components and/or process steps are added to the process workflows embodied herein. In some instances, a substrate is first cleaned, for example, using a piranha solution. An example of a cleaning process includes soaking a substrate in a piranha solution (e.g., 90% H₂SO₄, 10% H₂O₂) at an elevated temperature (e.g., 120° C.) and washing (e.g., water) and drying the substrate (e.g., nitrogen gas). The process optionally includes a post piranha treatment comprising soaking the piranha treated substrate in a basic solution (e.g., NH₄OH) followed by an aqueous wash (e.g., water). In some instances, a surface of a structure is plasma cleaned, optionally following the piranha soak and optional post piranha treatment. An example of a plasma cleaning process comprises an oxygen plasma etch. In some instances, the surface is deposited with an active functionalization agent following by vaporization. In some instances, the substrate is actively functionalized prior to cleaning, for example, by piranha treatment and/or plasma cleaning.

The process for surface functionalization optionally comprises a resist coat and a resist strip. In some instances, following active surface functionalization, the substrate is spin coated with a resist, for example, SPR™ 3612 positive photoresist. The process for surface functionalization, in various instances, comprises lithography with patterned functionalization. In some instances, photolithography is performed following resist coating. In some instances, after lithography, the surface is visually inspected for lithography defects. The process for surface functionalization, in some instances, comprises a cleaning step, whereby residues of the substrate are removed, for example, by plasma cleaning or etching. In some instances, the plasma cleaning step is performed at some step after the lithography step.

In some instances, a surface coated with a resist is treated to remove the resist, for example, after functionalization and/or after lithography. In some cases, the resist is removed with a solvent, for example, with a stripping solution comprising N-methyl-2-pyrrolidone. In some cases, resist stripping comprises sonication or ultrasonication. In some instances, a resist is coated and stripped, followed by active functionalization of the exposed areas to create a desired differential functionalization pattern.

In some instances, the methods and compositions described herein relate to the application of photoresist for the generation of modified surface properties in selective areas. In some instances, the application of the photoresist relies on the fluidic properties of the surface defining the spatial distribution of the photoresist. Without being bound by theory, surface tension effects related to the applied fluid may define the flow of the photoresist. For example, surface tension and/or capillary action effects may facilitate drawing of the photoresist into small structures in a controlled fashion before the resist solvents evaporate. In some instances, resist contact points are pinned by sharp edges, thereby controlling the advance of the fluid. The underlying structures may be designed based on the desired flow patterns that are used to apply photoresist during the manufacturing and functionalization processes. A solid organic layer left behind after solvents evaporate may be used to pursue the subsequent steps of the manufacturing process. Structures may be designed to control the flow of fluids by facilitating or inhibiting wicking effects into neighboring fluidic paths. For example, a structure is designed to avoid overlap between top and bottom edges, which facilitates the keeping of the fluid in top structures allowing for a particular disposition of the resist. In an alternative example, the top and bottom edges overlap, leading to the wicking of the applied fluid into bottom structures. Appropriate designs may be selected accordingly, depending on the desired application of the resist.

In some instances, a structure described herein has a surface that comprises a material having thickness of at least or at least 0.1 nm, 0.5 nm, 1 nm, 2 nm, 5 nm, 10 nm or 25 nm that comprises a reactive group capable of molecular sensors. Exemplary materials include, without limitation, gold, glass and silicon, such as silicon dioxide and silicon nitride. In some cases, exemplary surfaces include nylon and PMMA.

In some instances, electromagnetic radiation in the form of UV light is used for surface patterning. In some instances, a lamp is used for surface patterning, and a mask mediates exposure locations of the UV light to the surface. In some instances, a laser is used for surface patterning, and a shutter opened/closed state controls exposure of the UV light to the surface. The laser arrangement may be used in combination with a flexible structure that is capable of moving. In such an arrangement, the coordination of laser exposure and flexible structure movement is used to create patterns of one or more agents having differing nucleoside coupling capabilities.

Described herein are surfaces for molecular sensing that are reusable. After loading of charge sensors, a surface may be bathed, washed, cleaned, baked, etched, or otherwise functionally restored to a condition suitable for subsequent molecular sensing. The number of times a surface is reused and the methods for recycling/preparing the surface for reuse vary depending on subsequent applications. Surfaces prepared for reuse are in some instances reused about 1, 2, 3, 5, 10, 20, 50, 100, 1,000 or more times. Surfaces prepared for reuse are in some instances reused at least 1, 2, 3, 5, 10, 20, 50, 100, 1,000 or more times. In some instances, the remaining “life” or number of times a surface is suitable for reuse is measured or predicted.

In some instances, layers of a device are integrated into a solid support. In some instances, layers comprise electrodes or are configured for use as electrodes. Devices in some instances comprise at least 2, 3, 4, 5, 6, 10, 20, or more electrodes per device. In some instances, electrodes are configured as sources, drains, or gates. In some instances, layers comprise a metal oxide layer. In some instances, layers comprise a metal oxide layer comprising a continuous metal layer beneath it. Electrodes in some instances comprise at least one conductor, and are fabricated of materials well known in the art. In some instances, electrodes comprise at least one conductor and one or more insulators or semi-conductors. In some instances electrodes comprise platinum, titanium, or titanium nitride. In some instances electrodes comprise at least 20%, 30%, 40%, 50%, 60%, 70%, 80%, 90%, or at least 95%, of one or more of platinum, titanium, or titanium nitride. Materials in some instances comprise metals, non-metals, mixed-metal oxides, nitrides, carbides, silicon-based materials, or other material. In some instances, metal oxides include TiO₂, Ta₂O₅, IrO₂, RuO₂, RhO₂, Nb₂O₅, Al₂O₃, BaO, Y₂O₃, HfO₂, SrO or other metal oxide known in the art. In some instances, metal carbides include TiC, WC, ThC₂, ThC, VC, W₂C, ZrC, HfC, NbC, TaC, Ta₂C, or other metal carbide known in the art. In some instances, metal nitrides include GaN, InN, BN, Be₃N₂, Cr₂N, MoN, Si₃N₄, TaN, Th₂N₂, VN, ZrN, TiN, HfN, NbC, WN, TaN, or other metal nitride known in the art. In some instances, a device disclosed herein is manufactured with a combination of materials listed herein or any other suitable material known in the art. In some instances, layers described herein are coated with an additional metal. In some instances, layers described herein are coated with an additional material which is configured for attachment of a nanowire. In some instances, one or more layers are coated with gold. In some instances, one or more electrodes are coated in gold. In some instances, gold is deposited using direct, thermal evaporation. In some instances, gold is deposited using direct, thermal evaporation at a fixed angle. In some instances, gold is deposited using electrodeposition. In some instances, the thickness of the gold layer is about 10, 25, 30, 40, 50, 60, or about 75 angstroms. In some instances, the thickness of the gold layer is at least 10, 25, 30, 40, 50, 60, or at least 75 angstroms. In some instances, the thickness of the gold layer is no more than 10, 25, 30, 40, 50, 60, or no more than 75 angstroms. In some instances, the gold layer is added to a layer comprising another metal. In some instances, the gold layer is added to a layer comprising titanium.

In some instances, devices are contacted with one or more charge sensors. In some instances, charge sensors attach to one or more electrodes. In some instances charge sensors span at least two electrodes. Such charge sensors in some instances are attached to tether. In some instances the tether is further attached to a molecular sensor. In some instances, charge sensors comprise nanowires. In some instances, the nanowire comprises nucleic acids. In some instances, charge sensors (such as nucleic acids) are loaded onto the device surface. Loading in some instances comprises one or more of steps: (a) applying a voltage to the electrodes to attract the nucleic strands to the electrodes; (b) monitoring a current path between electrodes to determine if a nanowire has bridged two electrodes; and (c) when the current spikes (e.g., contact is made) the voltage is turned off so that no more DNA is attracted. In some instances, loading voltages are about 10-100V, 10-50V, 10-25V, 10-75V, or 25-100V. In some instances, the loading voltage is about 10 V, 15 V, 20 V, 25 V, 30 V, 40 V, 50 V, 60 V, 70 V, 75 V, 80 V, 90 V, or 100 V. In some instances, loading voltages are applied for no more than about 0.01, 0.05, 1, 2, 3, 4, 5, 6, 7, 8, 9, 10, 15, 20, 25, 30, 40, 50, 60, 90, or no more than 120 seconds.

Provided herein are devices comprising a surface. In some instances, the surface is modified to support molecular sensing at predetermined locations. In some instances, surfaces of devices for molecular sensing provided herein are fabricated from a variety of materials capable of modification to support molecular sensing. In some cases, the devices are sufficiently conductive, e.g., are able to form uniform electric fields across all or a portion of the devices. Devices described herein may comprise a flexible material. Exemplary flexible materials include, without limitation, modified nylon, unmodified nylon, nitrocellulose, and polypropylene. Devices described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Devices disclosed herein may be fabricated from a material comprising silicon, polystyrene, agarose, dextran, cellulosic polymers, polyacrylamides, polydimethylsiloxane (PDMS), glass, or any combination thereof. In some cases, devices disclosed herein are manufactured with a combination of materials listed herein or any other suitable material known in the art.

Devices described herein may comprise material having a range of tensile strength. Exemplary materials having a range of tensile strengths include, but are not limited to, nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for molecular sensing that is in the form of a flexible material capable of being stored in a continuous loop or reel, such as a tape or flexible sheet.

Young's modulus measures the resistance of a material to elastic (recoverable) deformation under load. Exemplary materials having a range of Young's modulus stiffness include, but are not limited to, nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load. In some instances, a solid support described herein has a surface with a flexibility of at least nylon.

In some cases, devices disclosed herein comprise a silicon dioxide base and a surface layer of silicon oxide. Alternatively, the devices may have a base of silicon oxide. Surface of the devices provided here may be textured, resulting in an increase overall surface area for molecular sensing. Devices disclosed herein in some instances comprise at least 5%, 10%, 25%, 50%, 80%, 90%, 95%, or 99% silicon. Devices disclosed herein in some instances are fabricated from silicon on insulator (SOI) wafer.

The structure may be fabricated from a variety of materials, suitable for the methods and compositions of the invention described herein. In instances, the materials from which the substrates/solid supports of the comprising the invention are fabricated exhibit a low level of polynucleotide binding. In some situations, material that are transparent to visible and/or UV light can be employed. Materials that are sufficiently conductive, e.g. those that can form uniform electric fields across all or a portion of the substrates/solids support described herein, can be utilized. In some instances, such materials may be connected to an electric ground. In some cases, the substrate or solid support can be heat conductive or insulated. The materials can be chemical resistant and heat resistant to support chemical or biochemical reactions. For flexible materials, materials of interest can include: nylon, both modified and unmodified, nitrocellulose, polypropylene, and the like.

For rigid materials, specific materials of interest include: glass; fuse silica; silicon, plastics (for example polytetrafluoroethylene, polypropylene, polystyrene, polycarbonate, and blends thereof, and the like); metals (for example, gold, platinum, and the like). The structure can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

In some instances, a substrate disclosed herein comprises a computer readable material. Computer readable materials include, without limitation, magnetic media, reel-to-reel tape, cartridge tape, cassette tape, flexible disk, paper media, film, microfiche, continuous tape (e.g., a belt) and any media suitable for storing electronic instructions. In some cases, the substrate comprises magnetic reel-to-reel tape or a magnetic belt. In some instances, the substrate comprises a flexible printed circuit board.

Structures described herein may be transparent to visible and/or UV light. In some instances, structures described herein are sufficiently conductive to form uniform electric fields across all or a portion of a structure. In some instances, structures described herein are heat conductive or insulated. In some instances, the structures are chemical resistant and heat resistant to support a chemical reaction. In some instances, the substrate is magnetic. In some instances, the structures comprise a metal or a metal alloy. Structures described herein may be integrated into a rack, such as a rack unit in a rack server described herein.

Molecular Sensing

Devices, systems, and methods described herein are useful for molecular sensing. In some instances, an analyte interacts with a molecular sensor in electrical communication with one or more electrodes. In some instances, interaction with the analyte results in a change in voltage, current, or resistance which is detectable at the device (signal or signal pattern). In some instances, the analyte comprises nucleotide triphosphates. In some instances, one or more nucleotide triphosphates generate a unique signal (are distinguishable). In some instances, each type of nucleotide triphosphate correlates to the identity of a unique base. By repeating such measurements to generate one or more signals, the sequence or identity of nucleoside in a nucleic is in some instances determined. In some instances, the molecular sensor comprises a polymerase. In some instances, binding of a nucleotide triphosphate to the polymerase generates a measurable signal within a device described herein. In some instances, the one or more nucleotide triphosphates comprise non-canonical or unnatural amino acids. In some instances, nucleotide triphosphates comprise an unnatural or non-canonical bases. In some instances, unnatural or non-canonical bases are configured to generate unique signals or signal patterns. Such signals in some instances are measured between one or more electrodes.

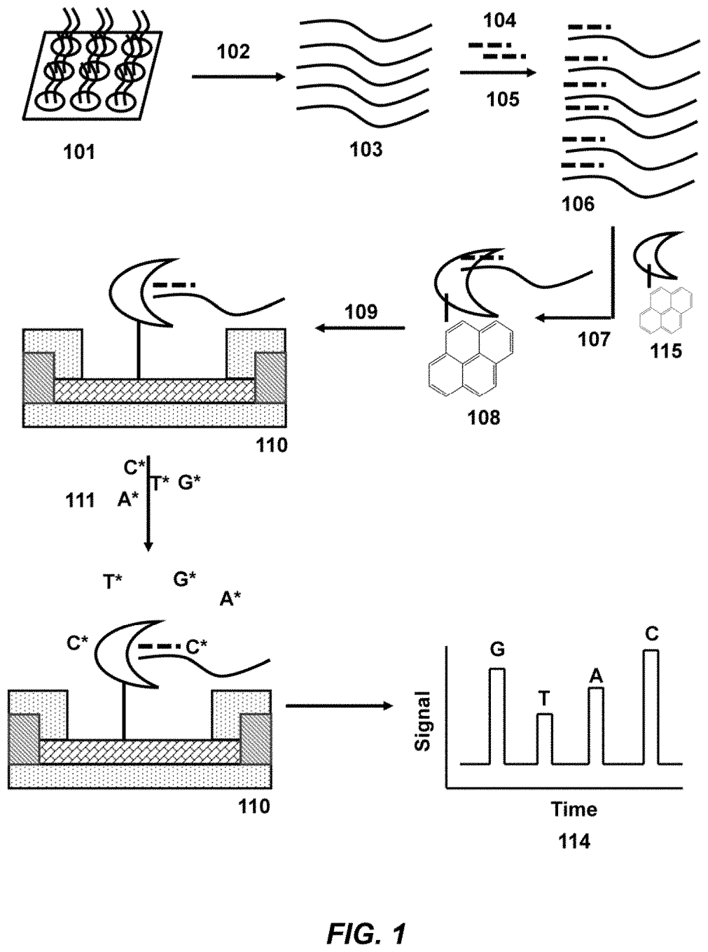

In an exemplary arrangement, a method described herein is provided in FIG. 1. In some instances, a method comprises any one of the steps in FIG. 1. Polynucleotides 103 attached (and/or synthesized) from a surface 101 are cleaved 102. Primers 104 are added 105, followed by a molecular sensor (e.g., polymerases) conjugated to a sensor-binding moiety 115 (graphene binder shown in FIG. 1, via optionally linker) to form a ternary complex 108. The ternary complex 108 is then contacted 109 with a graphene device 110 to bind the ternary complex 108 to a graphene layer. Charge modulating nucleotides (CMNs, indicated with base letter and an asterisk) 111 are added, which extend the primer of the ternary complex. Changes in current resulting from incorporation of CMNs 111 produce a signal which can be measured 114, thus identifying the incorporated base. In some instances, a charge sensor comprises a graphene layer, and a molecular sensor comprises a polymerase.

The method can include one or more steps of (a) providing a polymerase attached to a solid support charge sensor; (b) contacting the polymerase with a mixture of nucleotides; (c) detecting the incorporation of the nucleotides via the charge sensor; (d) repeating steps (b) and (c) using the polymerase, the template nucleic acid, and a second mixture of nucleotides; and (e) comparing the first and second signal patterns to determine the sequence of the template nucleic acid. In some instances, the mixture in (b) includes different types of nucleotides. In some instances, a first type of the nucleotide is in a distinguishable state compared to the other types of nucleotides in the mixture of (b). In some instances, a second type of the nucleotides is not in the distinguishable state compared to the other types of nucleotides in the mixture of (b). In some instances, the polymerase incorporates nucleotides from the mixture in (b) into a nascent strand against a template nucleic acid strand. In some instances, detecting the incorporation of the nucleotides via the charge comprises the first type of the nucleotides producing a signal that is unique compared to signals produced by other nucleotides in the mixture, thereby acquiring a first signal pattern. In some instances, repeating steps (b) and (c) using the polymerase comprises the second type of the nucleotides being in a distinguishable state compared to the other types of nucleotides in the second mixture. In some instances, repeating steps (b) and (c) using the polymerase comprises the first type of the nucleotides not being in the distinguishable state compared to the other types of nucleotides in the second mixture, thereby acquiring a second signal pattern.

Also provided is a method of nucleic acid sequencing that includes steps of (a) providing a polymerase attached to a solid support charge sensor; (b) contacting the polymerase with a mixture of nucleotides; (c) detecting the incorporation of the nucleotides via the charge sensor; (d) repeating steps (b) and (c) using the polymerase, the template nucleic acid, and a second mixture of nucleotides, wherein; and (e) comparing the first and second signal patterns to determine the sequence of the template nucleic acid. In some instances, the mixture in (b) includes different types of nucleotides. In some instances, a first two types of the nucleotides are in a first distinguishable state compared to a second two types of the nucleotides in the mixture of (b). In some instances, the polymerase incorporates nucleotides from the mixture in (b) into a nascent strand against a template nucleic acid strand. In some instances, detecting the incorporation of the nucleotides via the charge sensor comprises the first two types of the nucleotides producing a signal that distinguished from signals produced by second two types of the nucleotides in the mixture, thereby acquiring a first signal pattern. In some instances, repeating steps (b) and (c) using the polymerase one of the first two types of the nucleotides being in a distinguishable state compared to the other of the first two types of the nucleotides in the second mixture, thereby acquiring a second signal pattern.

In some instances, one or more non-natural nucleotide that are present in a mixture will produce a signal change having an inverted polarity compared to other nucleotides in the mixture. Alternatively or additionally, one or more non-natural nucleotide that is used in the mixture will produce a delay in nucleotide incorporation or reduced rate of incorporation. Alternatively or additionally, one or more non-natural nucleotide that is used in the method will produce a significantly altered signal height. These signal parameters can be detected in order to distinguish the nucleotides in a template nucleic acid to which the non-natural nucleotides complement during polymerase activity. In some instances, method of using a device described herein comprises one or more steps of providing an analyte; reacting, binding, or other allowing the analyte to interact with the sensor; and measuring an electrical signal generated from the sensor. In some instances, method of using a device described herein comprises one or more steps of providing at least one nucleotide, at least one template (nucleic acid), and at least one primer; extending the primer by the at least one nucleotide; and measuring an electrical signal generated from the polymerase. In some instances, electrical signals are analyzed to establish the identity of the at least one nucleotide incorporated by a polymerase. In some instances, at least one nucleotide comprises a terminator which is configured to prevent chain extension. In some instances, methods described herein are used to sequence at least 10, 20, 50, 100, 200, 300, 400, 500, 600, 700, 750, 800, 900, 1000, or more than 1000 bases. In some instances, methods described herein are used to sequence about 10, 20, 50, 100, 200, 300, 400, 500, 600, 700, 750, 800, 900, 1000, 1500, or about 2000 bases. In some instances, methods described herein are used to sequence 10-1000, 20-1000, 50-1000, 100-1000, 50-2000, 25-500, 25-200, 50-500, or 50-750 bases. In some instances, nucleotides comprise charge modulating nucleotides.

Interactions between a molecular sensor and an analyte (e.g., a polymerase and a nucleotide triphosphate) may generate detectable signals. In some instances the analyte comprises a charge modulating nucleotide. In some instances, a non-natural moiety or modification that is present in the non-natural nucleotide(s) produces a change in polymerase conformation (compared to the conformation produced by a nucleotide that lacks the moiety or modification) thereby producing a unique signature in one or more signal parameter detected by a charge sensor to which the polymerase is attached. Exemplary signal parameters include, but are not limited to, signal duration, signal height, signal rise time, signal fall time, signal polarity, signal noise, signal shape, and the like. In some instances, methods described herein utilize a mixture of four different types of nucleotide triphosphates in which one of the nucleotide triphosphate types is present in a substantially lower amount or concentration (e.g., the ‘low’ abundance nucleotide) compared to the other three types (e.g., the ‘high’ abundance nucleotides). As a result, incorporation of the low nucleotide will be detectable as a relative delay or decreased incorporation rate. This signature in some instances is exploited to identify the location in the template of the nucleotide type that complements the low nucleotide. Several sequencing runs in some instances are completed for the same template, wherein each run is carried out with a different nucleotide in the low state. The signal patterns from the different runs in some instances are compared to determine the sequence of the template. In some instances, the method comprises use of 3 high-1 low mixture of nucleotide triphosphates. Other ratios of high to low are also described herein. mixtures as well including, for example, a one high-three low mixture, or a two high-two low mixture. Further useful configurations of mixtures with regard to using nucleotides having different concentrations are set forth in U.S. Pat. No. 7,556,922, which is incorporated herein by reference. In particular embodiments the template nucleic acid is circular. The use of a circular template in some instances provides a convenient format for repeated sequencing runs since the polymerase need not be replaced and can instead make multiple laps around the template, each lap being effectively a repeated sequencing of the template. In some instances that utilize a circular template, the polymerase includes a 5′ exonuclease activity to digest a nucleic acid strand that is to be displaced from the circular template when the polymerase proceeds multiple times around the template. Whether the template is linear or circular, a different primer in some instances is used for different sequencing runs carried out on the same template. The different primers in some instances are designed to hybridize at different locations on the template. As such, each of the runs start at a different location in the template, but there is in some instances substantial overlap between the portions of the template that are sequenced in each run. The signal patterns resulting from each run in some instances is aligned based on the expected start sites for each run in order to facilitate sequence calling and error checking. A charge sensor that is used in a method set forth herein in some instances detects nucleotide incorporation by polymerase via a field effect using a SWNT FET, nanowire FET, FinFET, trigate FET, tunneling FET, or another field sensitive device. In some instances, the sensor is magnetic, electrochemical, or nanoelectromechanical. In some instances, each nucleotide triphosphate generates a distinguishable state. In some instances, distinguishable states applies to a particular type of nucleotide triphosphate having a characteristic or property that manifests uniquely under a detection condition compared to other nucleotide triphosphates. Exemplary distinguishable states include, but are not limited to, being present in a quantity or concentration that is substantially less than the quantity or concentration of the other types of nucleotide triphosphates in the mixture, being present in a quantity or concentration that is substantially greater than the quantity or concentration of the other types of nucleotide triphosphates in the mixture, having a chemical moiety or modification that is not present on other types of nucleotide triphosphates in the mixture, or lacking a chemical moiety or modification that is present on other types of nucleotide triphosphates in the mixture. The distinguishable state can manifest when the nucleotide type interacts with a polymerase. In some instances, signals are detected from conformational changes, such as the appearance, disappearance, or alteration of a detectable signal from a molecule in response to a change in the structure, shape or arrangement of parts of the molecule. For example, the signal change can be due to a change in the interaction of a label with a first portion of the molecule to interact with a second portion of the molecule.

Detectable signals may comprise changes to current, resistance, or voltage. In some instances, the detectable signal comprises a change in current. In some instances, the change in current is 1 nanoamp to 100 picoamps, 1 nanoamp to 50 picoamps, 1 nanoamp to 25 picoamps, 1 nanoamp to 10 picoamps, 1 nanoamp to 1 picoamp, 1 nanoamp to 500 nanoamps, 1 nanoamp to 250 nanoamps, 1 nanoamp to 100 nanoamps, 100 nanoamps to 100 picoamps, 100 nanoamps to 10 picoamps, 100 nanoamps to 1 picoamp, 100 nanoamps to 500 nanoamps, 500 nanoamps to 100 picoamps, 500 nanoamps to 50 picoamps, 500 nanoamps to 10 picoamps, 1 picoamp to 100 picoamps, 10 picoamps to 100 picoamps, or 250 nanoamps to 750 nanoamps. In some instances, a detectable signal is measured as a change in signal relative to a background signal (e.g., absence of analyte). In some instances, the change in current is 1.01-3, 1.01-2.75, 1.01-2.50, 1.01-2.25, 1.01-2, 1.01-1.95, 1.01-1.75, 1.01-1.5, 1.01-1.25, 1.25-3. 1.35-3, 1.5-3, 2-3, or 2.5-3 relative to a background signal.

Methods described herein may allow for rapid analysis of analytes. In some instances, 1-200, 1-500, 1-300, 1-150, 1-100, 10-500, 10-300, 50-300, 50-200, 100-200, or 150-400 biomolecules are analyzed per second. In some instances, at least 25, 50, 75, 100, 125, 150, 175, 200, 225, 250, 275, 300, 350, 400, 450, 500, or at least 600 biomolecules are analyzed per second. In some instances, the biomolecule comprises a nucleotide or variant thereof. In some instances, the biomolecule comprises a charge modulating nucleotide (CMN).

Analyte may comprise nucleotides. In some instances, an analyte comprises a charge modulating nucleotide. In some instances, the modification comprises a modification to the base (nucleobase) of the CMN, such as a modification to a C, T, G, or C base. An exemplary CMN is depicted in FIG. 3. In some instances, the modification comprises a deaza or halogen modified base. In some instances, the modification comprises a 7-deaza or 8-bromo modified base. In some instances, the CMN comprises a modification to the 5′ position. In some instances, the modification comprises a modification to a 5′ polyphosphate or chemical variant thereof. In some instances, the modification comprises a thiolated or bromated phosphate. In some instances, the polyphosphate comprises at least 3, 4, 5, 6, 8, or 10 phosphates or variants thereof. In some instances, the modification comprises a modification to a terminal 5′ polyphosphate or chemical variant thereof. In some instances, the modification comprises a polymer. In some instances, the polymer comprises one or more of a nucleic acid chain, a peptide chain, a polysaccharide, a lipid, a synthetic polymer, and a dendrimer. In some instances, the nucleic acid chain comprises at least 5, 10, 15, 20, 25, 30, 40, 50, 60, 75, 100, 125, 150, 175, 200, 300, 400, 500, 600, 700, 800, 900, 1000, 2000, 2500, 3000, or at least 5000 bases. In some instances, the nucleic acid chain comprises no more than 5, 10, 15, 20, 25, 30, 40, 50, 60, 75, 100, 125, 150, 175, 200, 300, 400, 500, 600, 700, 800, 900, 1000, 2000, 2500, 3000, or no more than 5000 bases. In some instances, the nucleic acid chain comprises about 5, 10, 15, 20, 25, 30, 40, 50, 60, 75, 100, 125, 150, 175, 200, 300, 400, 500, 600, 700, 800, 900,1000, 2000, 2500, 3000, or about 5000 bases. In some instances, the nucleic acid chain comprises 25-300, 25-500, 25-400, 25-300, 25-250, 50-500, 50-300, 75-300, 75-250, 75-200, 100-500, 125-500, 200-500, 300-500, 10-250, 25-5000, 50-5000, 100-5000, 200-5000, 500-5000, 1000-5000, 2000-5000 or 10-5000 bases.

In some instances, the nucleic acid chain is branched (dendrimeric). In some instances, the nucleic acid chain comprises a secondary structure. In some instances, the secondary structure comprises one or more of a hairpin, a loop, a helix, a G-quadraplex, and an I-motif. In some instances, the nucleic acid chain comprises a single strand, double strand, or triple strand. In some instances, the nucleic acid chain comprises at least one charge modulating chemical modification. In some instances, the at least one charge modulating chemical modification increases the charge of the CMN relative to an unmodified nucleotide. In some instances, the at least one charge modulating chemical modification comprises one or more of an amine, an alkylamine, a guanidinium, a quaternary amine, an imidazolium, a pyridinium, and a pyrrolidinium. In some instances, the at least one charge modulating chemical modification decreases the charge of the CMN relative to an unmodified nucleotide. In some instances, the at least one charge modulating chemical modification comprises one or more of a phosphate, a phosphite, a sulfonate, a sulfite, a carboxylate, a xanthate, a thiocarboxylic acid, a boranophosphonate, and a boric acid. In some instances, the nucleic acid chain comprises at least one sugar-modified nucleotide. In some instances, the sugar-modified nucleotide comprises a deoxy or dideoxy nucleotide. In some instances, the nucleic acid chain comprises a DNA-DNA, DNA-RNA, or DNA-PNA hybrid.

A nucleic acid chain may comprise a phosphate modification. In some instances, the phosphate modification comprises a hydrophobic group. In some instances, the hydrophobic group comprises a straight or branched alkyl chain. In some instances, the hydrophobic group comprises a C₅-C₅₀ aliphatic chain. In some instances, the phosphate modification comprises a hydrophilic group. In some instances, the hydrophilic group comprises polyethylene glycol (PEG). In some instances, the polyethylene glycol comprises a molecular weight of 1000-100,000, 100-500,000, 100-250,000, 100-75,000, 100-50,000, 100-25,000, 100-10,000, 100-7500, 100-5000, 100-3000, or 100-2000 daltons. In some instances, the polyethylene glycol comprises a molecular weight of about 100, 200, 300, 500, 1000, 2000, 2500, 3000, 5000, 7500, 10,000, 25,000, 50,000, 75,000, 100,000, 250,000, or 50,000 daltons. In some instances, the polyethylene glycol comprises a molecular weight of at least about 100, 200, 300, 500, 1000, 2000, 2500, 3000, 5000, 7500, 10,000, 25,000, 50,000, 75,000, 100,000, 250,000, or 50,000 daltons. In some instances, the polyethylene glycol comprises a molecular weight of at most about 100, 200, 300, 500, 1000, 2000, 2500, 3000, 5000, 7500, 10,000, 25,000, 50,000, 75,000, 100,000, 250,000, or 50,000 daltons. In some instances, the polyethylene glycol comprises 10-600 monomers. In some instances, the polyethylene glycol comprises about 10, 20, 30, 40, 50, 60, 70, 80, 90, 100, 200, 300, 400, 500, or 600 monomers. In some instances, the polyethylene glycol comprises at least about 10, 20, 30, 40, 50, 60, 70, 80, 90, 100, 200, 300, 400, 500, or 600 monomers. In some instances, the polyethylene glycol comprises at most about 10, 20, 30, 40, 50, 60, 70, 80, 90, 100, 200, 300, 400, 500, or 600 monomers. In some instances, the peptide chain is 1-100 amino acids in length. In some instances, the peptide chain is about 1, 2, 3, 4, 5, 10, 20, 30, 40, 50, 60, 70, 80, 90, or 100 amino acids in length. In some instances, the peptide chain is at least about 1, 2, 3, 4, 5, 10, 20, 30, 40, 50, 60, 70, 80, 90, or 100 amino acids in length. In some instances, the peptide chain is at most about 1, 2, 3, 4, 5, 10, 20, 30, 40, 50, 60, 70, 80, 90, or 100 amino acids in length. In some instances, the CMN comprises a charged small molecule. In some instances, the charged small molecule comprises one or more of a chelator, a dye, and a metal complex. In some instances, the metal complex comprises a ferrocene, Ru-dipy, and bis-cyclopentadienyl diiron.

Molecular sensors described herein may be conformationally labeled. In some instances, conformational labeling comprises at least one label that is responsive to a change in the structure of the molecule, a change in the shape of the molecule, or a change in the arrangement of parts of the molecule. The molecule is in some instances a polymerase, reverse transcriptase, exonuclease or other nucleic acid enzyme. The parts of the molecule can be, for example, atoms that change relative location due to rotation about one or more chemical bonds occurring in the molecular structure between the atoms. The parts of the molecule can be domains of a macromolecule such as those commonly known in the relevant art. In some instances, polymerases include domains referred to as the finger, palm and thumb domains. In the case of proteins the parts can be regions of secondary, tertiary or quaternary structure. The label(s) can be attached to the molecule, for example, via a covalent linkage. However, the label(s) need not be attached to the molecule, being, for example, located in proximity to the molecule. In particular embodiments, the label is not attached to a reactant or product of the molecule such as a nucleotide or nucleic acid. In some instances, a molecular sensor comprises a polymerase.

Provided herein are methods for nucleic acid sequencing. In some instances, a method comprises one or more of contacting a plurality of polynucleotides with at least one primer and at least one polymerase to form a plurality of ternary complexes, wherein the ternary complex comprising a graphene binder; detecting one or more bases of the polynucleotides, wherein detection occurs when the plurality of ternary complexes are bound to a graphene layer; removing the ternary complexes from the surface; and repeating previous steps to sequence the polynucleotides. In some instances, ternary complexes are attracted to the graphene layer by applying a positive charge to one or more electrodes buried underneath the graphene layer. The plurality of polynucleotides comprises at least 10,000, 50,000, 100,000 250,000, 300,000, 400,000, 500,000, 600,000, 700,000, 750,000, 800,000, 900,000, 1 million, 10 million, 100 million, 200 million, 500 million, or at least 750 million unique polynucleotides. The plurality of polynucleotides comprises about 10,000, 50,000, 100,000 250,000, 300,000, 400,000, 500,000, 600,000, 700,000, 750,000, 800,000, 900,000, 1 million, 10 million, 100 million, 200 million, 500 million, or at least 750 million unique polynucleotides. In some instances the plurality of polynucleotides are 50-30,000, 50-10,000, 50-1000, 50-750, 50-500, 50-400, 50-300, 50-200, 1000-30,000, 1000-20,000, 1000-10,000, 2000-5000, 2000-10,000, 5000-30,000, or 10,000-30,000 bases in length. In some instances, the plurality of polynucleotides are about 10, 50, 100, 200, 300, 400, 500, 750, 1000, 2000, 2500, 5000, 10,000, 20,000, or 30,000 bases in length.

Nucleic Acid-Based Information

Provided herein are devices, compositions, systems and methods for reading nucleic acid-based information (data). In a first step, a digital sequence encoding an item of information (e.g., digital information in a binary code for processing by a computer) is received. An encryption scheme is applied to convert the digital sequence from one or more symbols (e.g., a binary code) to a nucleic acid sequence. A surface material for nucleic acid extension, a design for loci for nucleic acid extension (e.g., arrangement spots), and reagents for nucleic acid synthesis are selected. The surface of a structure is prepared for nucleic acid synthesis. De novo polynucleotide synthesis is in some instances performed. The synthesized polynucleotides are stored and available for subsequent release, in whole or in part. In some instances, pools of pre-determined polynucleotides are assembled into larger polynucleotides which represent digital information. Once released, the polynucleotides, in whole or in part, are sequenced using the devices, systems, and methods described herein, subject to decryption to convert nucleic sequence back to digital sequence. The digital sequence is then assembled to obtain an alignment encoding for the original item of information. In some instances, polynucleotides are sequenced using the methods and devices described herein.

Nucleic acids encoding digital information may comprise error correction component. In some instances, the error correction component comprises an error correction code, such as a Reed-Solomon (RS) code, a LDPC code, a polar code, a turbo code. In some instances, the error correction code spreads the digital data to be stored over many polynucleotides. In some instances, spreading the data over a plurality of polynucleotides builds redundancy to correct for erasures (e.g., lost oligos). In some instances, the digital information can be recovered in the presence of errors. In some instances, the error correction component comprises a parity base. In some instances, the error correction component comprises an index sequence. In some instances, the index sequences define the location or address of the digital information encoded in the nucleic acid. In some instances, the index sequences define the source of the digital information. Nucleic acids encoding digital information in some instances comprise overlap with one or more nucleic acids in the same library or set. In some instances, the error correction component comprises an overlap or redundancy region. In some instances, algorithms are applied to sequenced nucleic acids to reduce errors. In some instances, error corrective algorithms comprise consensus sequencing, HEDGES (Hash Encoded, Decoded by Greedy Exhaustive Search), or other method.

Nucleic acids encoding for digital information may be stored in different media. In some instances, nucleic acids are stored as essentially dry or lyophilized powders. In some instances, nucleic acids are stored in buffers. In some instances, nucleic acids are stored on chips, wafers, or other silicon solid support. In some instances, nucleic acids are stored inside an organism (or population of organisms), such as a plasmid or genome.

Optionally, an early step of data storage process disclosed herein includes obtaining or receiving one or more items of information in the form of an initial code. Items of information include, without limitation, text, audio and visual information. Exemplary sources for items of information include, without limitation, books, periodicals, electronic databases, medical records, letters, forms, voice recordings, animal recordings, biological profiles, broadcasts, films, short videos, emails, bookkeeping phone logs, internet activity logs, drawings, paintings, prints, photographs, pixelated graphics, and software code. Exemplary biological profile sources for items of information include, without limitation, gene libraries, genomes, gene expression data, and protein activity data. Exemplary formats for items of information include, without limitation, .txt, .PDF, .doc, .docx, .ppt, .pptx, .xls, .xlsx, .rtf, .jpg, .gif, .psd, .bmp, .tiff, .png, and .mpeg. The amount of individual file sizes encoding for an item of information, or a plurality of files encoding for items of information, in digital format include, without limitation, up to 1024 bytes (equal to 1 KB), 1024 KB (equal to 1 MB), 1024 MB (equal to 1 GB), 1024 GB (equal to 1 TB), 1024 TB (equal to 1 PB), 1 exabyte, 1 zettabyte, 1 yottabyte, 1 xenottabyte or more. In some instances, an amount of digital information is at least 1 gigabyte (GB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 gigabytes. In some instances, the amount of digital information is at least 1 terabyte (TB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 terabytes. In some instances, the amount of digital information is at least 1 petabyte (PB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 petabytes.

The solid support for molecular sensing as described herein comprises a high capacity for reading of data. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 petabytes. In some instances, the capacity of the solid support is between about 1 to about 10 petabytes or between about 1 to about 100 petabytes. In some instances, the capacity of the solid support is about 100 petabytes. In some instances, the data is stored as addressable arrays of packets as droplets. In some instances, the data is stored as addressable arrays of packets as droplets on a spot. In some instances, the data is stored as addressable arrays of packets as dry wells. In some instances, the addressable arrays comprise at least or about 1, 2, 3, 4, 5, 6, 7, 8, 9, 10, 20, 50, 100, 200, or more than 200 gigabytes of data. In some instances, the addressable arrays comprise at least or about 1, 2, 3, 4, 5, 6, 7, 8, 9, 10, 20, 50, 100, 200, or more than 200 terabytes of data. In some instances, an item of information is stored in a background of data. For example, an item of information encodes for about 10 to about 100 megabytes of data and is stored in 1 petabyte of background data. In some instances, an item of information encodes for at least or about 1, 10, 20, 30, 40, 50, 60, 70, 80, 90, 100, 150, 200, 300, 400, 500, or more than 500 megabytes of data and is stored in 1, 10, 20, 30, 40, 50, 60, 70, 80, 90, 100, 150, 200, 300, 400, 500, or more than 500 petabytes of background data.

Computer Systems

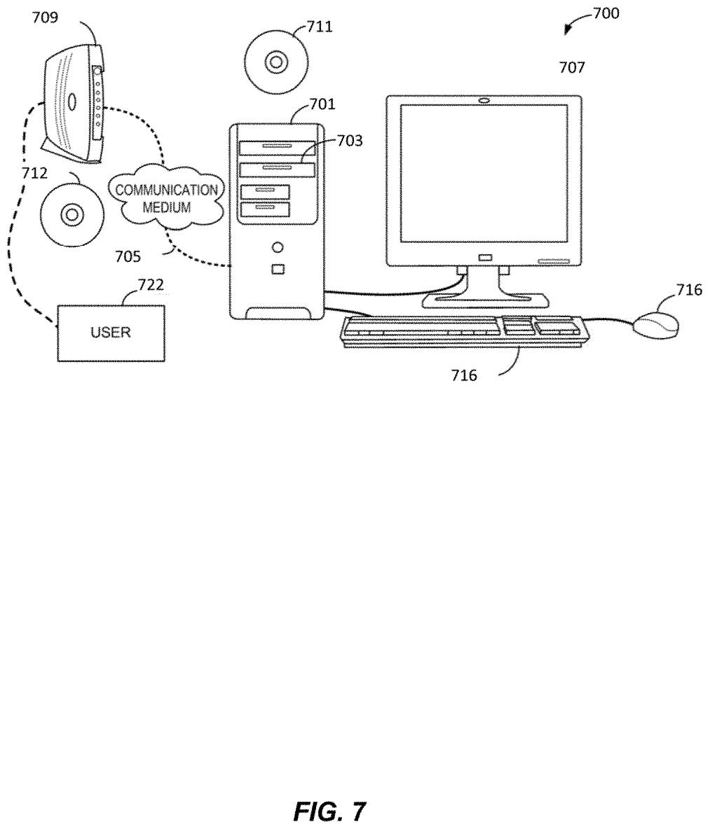

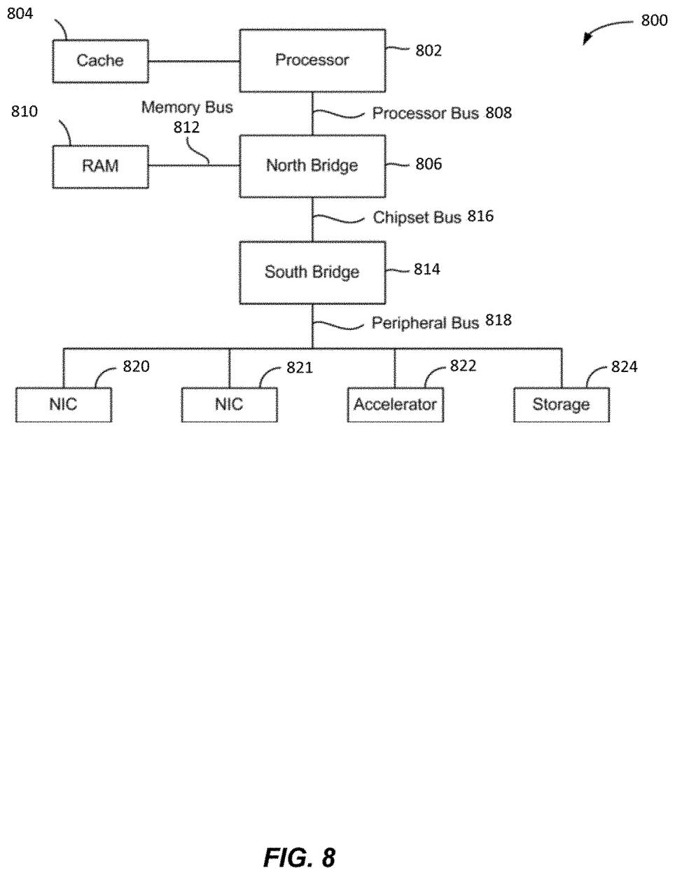

In various aspects, any of the systems described herein are operably linked to a computer and are optionally automated through a computer either locally or remotely. In various instances, the methods and systems of the invention further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. In some instances, the computer systems are programmed to interface between the user specified base sequence and the position of a material deposition device to deliver the correct reagents to specified regions of the substrate. As an example, a computer system, such as the system shown in FIG. 7 or FIG. 8, may be used for encoding data represented as a set of symbols to another set of symbols. For example, the data may be represented as numerical symbols, such as binary values of “0”s and “1”s and the computer system may execute a program comprising an error correction code (e.g., Reed-Solomon (RS) code, low-density parity-check (LDPC) code, Turbo code, etc.). In some instances, the computer system executes a program to convert the data to a plurality of nucleic acid sequences, convert a plurality of nucleic acid sequences to data, or both. In some examples, a program may be a machine learning algorithm. In some examples, the machine learning algorithm may determine a nucleotide base based on a signal (e.g., electrical signal, such as current or voltage).

A program may be executed on a computer system provided herein. In some instances, a program comprises a statistical algorithm or a machine learning algorithm. In some instances, an algorithm comprising machine learning (ML) is used to associate the signal (e.g., electrical currents/voltages) to the nucleoside monomer added to the polynucleotide. In some cases, the algorithm comprising ML may be trained with training data in order to associate the signal (e.g., electrical currents/voltages) to the nucleoside monomer added to the polynucleotide. In some cases, the algorithm comprises classical ML algorithms for classification and/or clustering (e.g., K-means clustering, mean-shift clustering, density-based spatial clustering of applications with noise (DBSCAN), expectation-maximization (EM) clustering, agglomerative hierarchical clustering, logistic regression, naïve Bayes, K-nearest neighbors, random forests or decision trees, gradient boosting, support vector machines (SVMs), or a combination thereof).

In some cases, the algorithm comprises a learning algorithm comprising layers, such as one or more neural networks. Neural networks may comprise connected nodes in a network, which may perform functions, such as transforming or translating input data. In some examples, the output from a given node may be passed on as input to another node. In some embodiments, the nodes in the network may comprise input units, hidden units, output units, or a combination thereof. In some cases, an input node may be connected to one or more hidden units. In some cases, one or more hidden units may be connected to an output unit. The nodes may take in input and may generate an output based on an activation function. In some embodiments, the input or output may be a tensor, a matrix, a vector, an array, or a scalar. In some embodiments, the activation function may be a Rectified Linear Unit (ReLU) activation function, a sigmoid activation function, or a hyperbolic tangent activation function. In some embodiments, the activation function may be a Softmax activation function. The connections between nodes may further comprise weights for adjusting input data to a given node (e.g., to activate input data or deactivate input data). In some embodiments, the weights may be learned by the neural network. In some embodiments, the neural network may be trained using gradient-based optimizations. In some cases, the gradient-based optimization may comprise of one or more loss functions. In some examples, the gradient-based optimization may be conjugate gradient descent, stochastic gradient descent, or a variation thereof (e.g., adaptive moment estimation (Adam)). In further examples, the gradient in the gradient-based optimization may be computed using backpropagation. In some embodiments, the nodes may be organized into graphs to generate a network (e.g., graph neural networks). In some embodiments, the nodes may be organized into one or more layers to generate a network (e.g., feed forward neural networks, convolutional neural networks (CNNs), recurrent neural networks (RNNs), etc.). In some cases, the neural network may be a deep neural network comprising of more than one layer.

In some cases, the neural network may comprise one or more recurrent layer. In some examples, the one or more recurrent layer may be one or more long short-term memory (LSTM) layers or gated recurrent unit (GRU), which may perform sequential data classification and clustering. In some embodiments, the neural network may comprise one or more convolutional layers. The input and output may be a tensor representing of variables or attributes in a data set (e.g., features), which may be referred to as a feature map (or activation map). In some cases, the convolutions may be one dimensional (1D) convolutions, two dimensional (2D) convolutions, three dimensional (3D) convolutions, or any combination thereof. In further cases, the convolutions may be 1D transpose convolutions, 2D transpose convolutions, 3D transpose convolutions, or any combination thereof. In some examples, one-dimensional convolutional layers may be suited for time series data since it may classify time series through parallel convolutions. In some examples, convolutional layers may be used for analyzing a signal or patterns in a signal (e.g., electrical currents/voltages) to the nucleoside monomer added to the polynucleotide.

The layers in a neural network may further comprise one or more pooling layers before or after a convolutional layer. The one or more pooling layers may reduce the dimensionality of the feature map using filters that summarize regions of a matrix. This may down sample the number of outputs, and thus reduce the parameters and computational resources needed for the neural network. In some embodiments, the one or more pooling layers may be max pooling, min pooling, average pooling, global pooling, norm pooling, or a combination thereof. Max pooling may reduce the dimensionality of the data by taking only the maximums values in the region of the matrix, which helps capture the significant feature. In some embodiments, the one or more pooling layers may be one dimensional (1D), two dimensional (2D), three dimensional (3D), or any combination thereof. The neural network may further comprise of one or more flattening layers, which may flatten the input to be passed on to the next layer. In some cases, the input may be flattened by reducing it to a one-dimensional array. The flattened inputs may be used to output a classification of an object (e.g., classification of signals (e.g., electrical currents/voltages) to a nucleoside monomer added to the polynucleotide, etc.). The neural networks may further comprise one or more dropout layers. Dropout layers may be used during training of the neural network (e.g., to perform binary or multi-class classifications). The one or more dropout layers may randomly set certain weights as 0, which may set corresponding elements in the feature map as 0, so the neural network may avoid overfitting. The neural network may further comprise one or more dense layers, which comprise a fully connected network. In the dense layer, information may be passed through the fully connected network to generate a predicted classification of an object, and the error may be calculated. In some embodiments, the error may be backpropagated to improve the prediction. The one or more dense layers may comprise a Softmax activation function, which may convert a vector of numbers to a vector of probabilities. These probabilities may be subsequently used in classifications, such as classifications of signals (e.g., electrical currents and/or voltages) to the nucleoside monomer added to the polynucleotide.

The computer system 700 illustrated in FIG. 7 may be understood as a logical apparatus that can read instructions from media 711 and/or a network port 705, which can optionally be connected to server 709 having fixed media 712. The system can include a CPU 701, disk drives 703, optional input devices such as keyboard 715 and/or mouse 716 and optional monitor 707. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 722.

FIG. 8 is a block diagram illustrating a first example architecture of a computer system that can be used in connection with example instances of the present invention. As depicted in FIG. 8, the example computer system can include a processor 802 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices. As illustrated in FIG. 8, a high speed cache 804 can be connected to, or incorporated in, the processor 802 to provide a high speed memory for instructions or data that have been recently, or are frequently, used by processor 802. The processor 802 is connected to a north bridge 806 by a processor bus 808. The north bridge 806 is connected to random access memory (RAM) 810 by a memory bus 812 and manages access to the RAM 810 by the processor 802. The north bridge 806 is also connected to a south bridge 814 by a chipset bus 816. The south bridge 814 is, in turn, connected to a peripheral bus 818. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 818. In some alternative architectures, the functionality of the north bridge can be incorporated into the processor instead of using a separate north bridge chip. In some instances, a system 800 can include an accelerator card 822 attached to the peripheral bus 818. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 824 and can be loaded into RAM 810 and/or cache 804 for use by the processor. The system 800 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention. In this example, system 800 also includes network interface cards (NICs) 820 and 821 connected to the peripheral bus for providing network interfaces to external storage, such as Network Attached Storage (NAS) and other computer systems that can be used for distributed parallel processing.

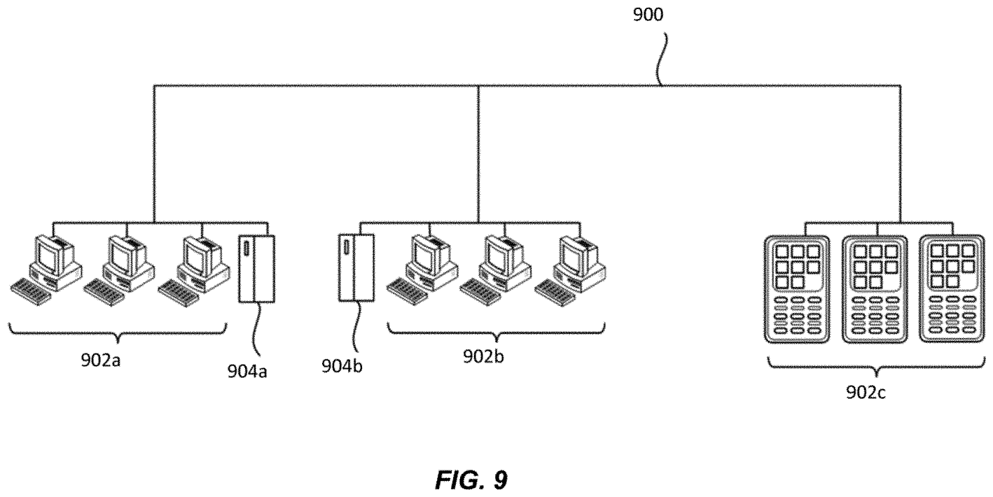

FIG. 9 is a diagram showing a network 900 with a plurality of computer systems 902a, and 902b, a plurality of cell phones and personal data assistants 902c, and Network Attached Storage (NAS) 904a, and 904b. In example embodiments, systems 902a, 902b, and 902c can manage data storage and optimize data access for data stored in Network Attached Storage (NAS) 904a and 904b. A mathematical model can be used for the data and be evaluated using distributed parallel processing across computer systems 902a, and 902b, and cell phone and personal data assistant systems 902c. Computer systems 902a, and 902b, and cell phone and personal data assistant systems 902c can also provide parallel processing for adaptive data restructuring of the data stored in Network Attached Storage (NAS) 904a and 904b. FIG. 9 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface.

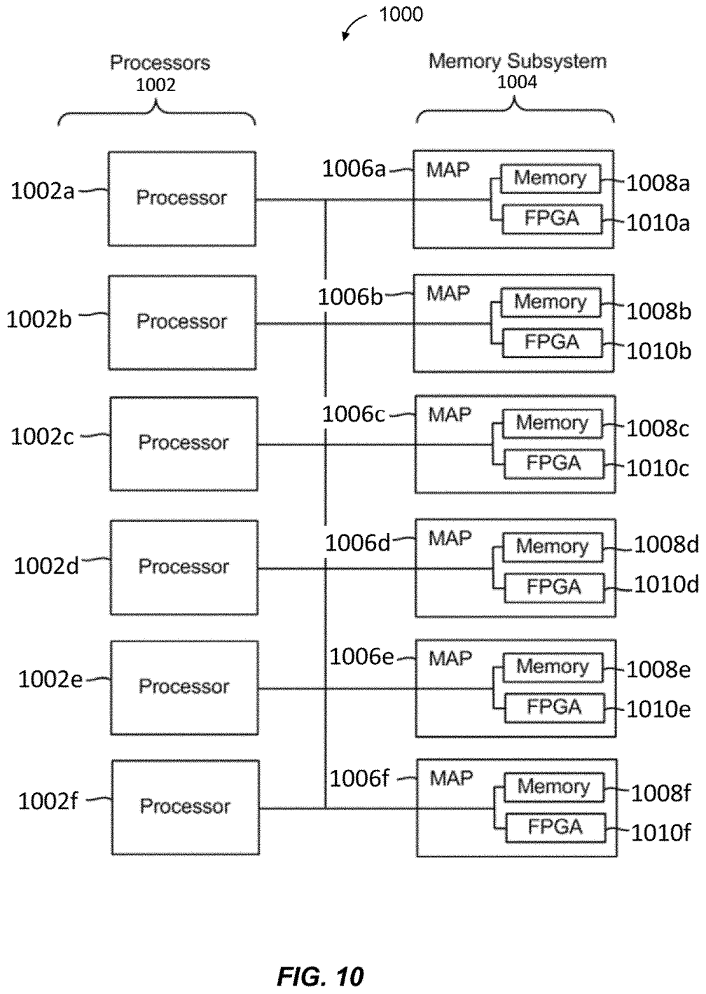

In some example embodiments, processors can maintain separate memory spaces and transmit data through network interfaces, back plane or other connectors for parallel processing by other processors. In other embodiments, some or all of the processors can use a shared virtual address memory space. FIG. 10 is a block diagram of a multiprocessor computer system 1000 using a shared virtual address memory space in accordance with an example embodiment. The system includes a plurality of processors 1002a-f that can access a shared memory subsystem 1004. The system incorporates a plurality of programmable hardware memory algorithm processors (MAPs) 1006a-f in the memory subsystem 1004. Each MAP 1006a-f can comprise a memory 1008a-f and one or more field programmable gate arrays (FPGAs) 1010a-f. The MAP provides a configurable functional unit and particular algorithms or portions of algorithms can be provided to the FPGAs 1010a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 1008a-f, allowing it to execute tasks independently of, and asynchronously from, the respective microprocessor 1002a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs), system on chips (SOCs), application specific integrated circuits (ASICs), or other processing and logic elements. For example, the Set Processor and Optimizer can be implemented with hardware acceleration through the use of a hardware accelerator card.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1

### Fabrication of a Graphene Device

Any array of addressable devices shown in FIG. 2 is constructed into a chip having a size of 4 to 16 mm², and a pitch distance of 50-1000 nm using the general methods of graphene FET fabrication described in U.S. Pat. No. 9,859,394, incorporated by reference in its entirety.

### Example 2

### Sequencing with a Graphene Device

A device of Example 1 is used to sequence nucleic acids. A device is contacted with a nucleic acid template, at least one primer which is configured to bind to the nucleic acid template, and a polymerase. The polymerase comprises a pyrene moiety linked to a Phi29 polymerase. The primer, sensor, and nucleic acid template form a ternary complex which is bound to the graphene surface of the device, A mixture of four nucleotides is contacted with the device and the template, wherein at least one nucleic acid generates a uniquely detectable signal from the device upon interaction with the polymerase. Optionally, after extension of the at least one primer by a nucleotide triphosphate and measurement of a signal corresponding to one of the nucleotide triphosphates, terminators are removed from the incorporated nucleotides. The process is repeated to establish the identity of each base added, thus determining the sequence of the nucleic acid template. Optionally, the ternary complexes are washed off the device and the device is reused.

### Example 3

### Fabrication of a Graphene Device with Buried Gate and Shield

Any array of addressable devices shown in FIG. 4A-4B is constructed into a chip having a size of 4 to 16 mm², and a pitch distance of 50-1000 nm using the general methods of graphene FET fabrication described in Example 1.

### Example 4

### Sequencing with a Graphene Device having a Buried Gate and Shield

A device of Example 3 is used to sequence nucleic acids using the general methods of Example 2 with modification. During ternary complex loading, a positive voltage is applied to the buried gate. This will attract negatively charged DNA (or other similar moieties) to towards the surface and the graphene. The shield layer, which is at ground potential, is configured with small openings that allow the positive potential from the gate to leak out to specific locations on the chip surface. The localization of the potential will concentrate the DNA (or similar moieties) to the locations of interest on the graphene. This promotes higher loading of the devices. During sensor operation, the gate can be used to modulate the graphene potential to maximize the signal change associated with molecular events occurring on or near the graphene layer.

### Example 5

### Edge Finger Device Fabrication

An array of devices having the general structure of FIGS. 12A-12C is fabricated by a) providing one or more base layers; b) depositing material to generate a second electrode; c) patterning the second electrode; d) optionally planarizing; e) depositing material to generate a passive layer; f) depositing material to generate a first electrode; g) patterning the first electrode; and h) isotropically etching the passive layer, such that the edges of the first electrode are undercut and the device comprises a nanogap of about 20 nm. Each electrode is fabricated from one or more of titanium, platinum, and titanium nitride. The passive layer comprises an oxide. Next, a layer of gold is deposited over the electrodes. After fabrication, the device is contacted with one or more nanowires comprising nucleic acids, wherein at least some of the nanowires bridge one or more first electrodes and second electrodes. Each nanowire comprises at least one biotin functional handle (optionally connected via a tether). Loading of the nanowires comprises (a) applying a voltage to the electrodes to attract the nucleic strands to the electrodes; (b) monitoring a current path between electrodes to determine if a nanowire has bridged two electrodes; and (c) when the current spikes (i.e., contact is made) turning off the voltage so that no more DNA is attracted. Next, nanowires are contacted with polymerases tethered to streptavidin to facilitate attachment of polymerases to the nanowires (e.g., similar to FIG. 11).

### Example 6

### Crossed Fingers Device

Following the general procedure or Example 5, an array of devices of FIGS. 13A-13B is fabricated. Devices are arrayed according to the general arrangement of FIG. 15.

### Example 7

### Crossed Fingers Device with Oxide Layer

Following the general procedure or Example 5, an array of devices of FIGS. 14A-14B is fabricated.

### Example 8

### Self-Aligned Finger Etch

Following the general procedure or Example 5, an array of devices of FIGS. 16A-16B is fabricated.

### Example 9

### Sequencing

A device of any of Examples 5-8 is used to sequence nucleic acids. A device is contacted with a nucleic acid template, at least one primer which is configured to bind to the nucleic acid template, and a mixture of four nucleotide triphosphates (and/or CMNs) is contacted with the device and the template, wherein at least one nucleic acid generates a uniquely detectable signal from the device upon interaction with the polymerase. After extension of the at least one primer by a nucleotide triphosphate and measurement of a signal corresponding to one of the nucleotide triphosphates, terminators are removed from the incorporated nucleotides. The process is repeated to establish the identity of each base added (e.g., A, T, C, or G), thus determining the sequence of the nucleic acid template.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for single molecule sensing comprising:
a. contacting a molecular sensor with at least one charge modulator, wherein contacting generates a change in current;
b. measuring the change in current; and
c. correlating the change in current with the presence or absence of the at least one charge modulator, thereby sensing the single molecule.

2. The method of claim 1, wherein the single molecule comprises a nucleic acid.

3.-4. (canceled)

5. The method of claim 1, wherein the charge sensor comprises a graphene-enabled field effect transistor (GeFET) or CMOS device.

6. The method of claim 1, wherein the molecular sensor is in electrical communication with a charge sensor.

7. The method of claim 1, wherein the molecular sensor comprises a polymerase.

8.-10. (canceled)

11. The method of claim 1, wherein the at least one charge modulator comprises a charge modulating nucleotide (CMN).

12. The method of claim 1, wherein contacting comprising incorporating a CMN into a polynucleotide primer.

13. The method of claim 11, wherein the CMN comprises at least one modification relative to a canonical nucleotide.

14.-45. (canceled)

46. The method of claim 1, wherein the change in current is 1 nanoamp to 100 picoamps.

47. (canceled)

48. The method of claim 1, wherein the change in current is at least 1.01-3 times the background current.

49. The method of claim 1, wherein steps a-c are repeated at least 50 times.

50. The method of claim 1, wherein method is configured to detect 1-200 single molecules per second.

51. A chemically-sensitive field effect transistor device for sensing single molecules comprising:
a solid support, wherein the solid support comprises a plurality of loci, wherein each loci comprises:
a graphene layer;
a gate electrode and a drain electrode, where the gate electrode and the drain electrode are in electrical communication via the graphene layer; and
at least one insulating layer, where the insulating layer is located between the gate electrode and the drain electrode;
wherein the loci have a pitch of 50-1000 nanometers.

52. The device of claim 51, wherein the device further comprises at least one ground shield.

53. (canceled)

54. The device of claim 51, wherein the device further comprises at least one buried gate.

55. The device of claim 52, wherein the at least one ground shield comprises an opening which permits electrical communication between the graphene layer and the least one buried gate.

56. The device of claim 51, wherein the graphene layer is approximately one atom thick.

57.-62. (canceled)

63. A method for single molecule polynucleotide sequencing comprising:
a) contacting a plurality of polynucleotides with at least one primer and at least one polymerase to form a plurality of ternary complexes, wherein the ternary complex comprising a graphene binder;
b) detecting one or more bases of the polynucleotides in real time, wherein detection occurs when the plurality of ternary complexes are bound to the graphene layer of the device of claim 51;
c) removing the ternary complexes from the surface; and
d) repeating steps a-c to sequence the polynucleotides.

64. The method of claim 63, wherein the graphene binder comprises an aromatic group.

65.-84. (canceled)

85. The method of claim 63, wherein detecting comprises measuring a change in current when a CMN is incorporated.

86.-213. (canceled)
